(19) United States
(12) Patent Application Publication
(10) Pub. No.: US 2022/0249239 A1
(43) Pub. Date: Aug. 11, 2022

(54) RESURFACING IMPLANT SYSTEMS AND METHODS FOR OSTEOCHONDRAL DEFECTS

(71) Applicant: Overture Resurfacing, LLC, New York, NY (US)

(72) Inventors: Riley J. Williams, New York, NY (US); Hyun Bae, Los Angeles, CA (US); Benjamin Arnold, San Diego, CA (US); Brian Bowman, Carlsbad, CA (US); Ian Fried, San Diego, CA (US)

(21) Appl. No.: 17/592,896

(22) Filed: Feb. 4, 2022

Related U.S. Application Data
(60) Provisional application No. 63/146,491, filed on Feb. 5, 2021.

(51) Int. Cl. A61F 2/38; A61F 2/30

(52) U.S. Cl. CPC A61F 2/3859; A61F 2/389; A61F 2/30771; A61F 2002/3093; A61F 2002/30841; A61F 2002/30934; A61F 2002/3895; A61F 2002/30878

(57) ABSTRACT

Exemplary arthroplasty systems and methods involve the implantation of a first implant having a convex bearing surface portion and a subchondral surface portion, and a second implant having a concave bearing surface portion and a subchondral surface portion, where the implants are configured for implantation into a joint of a patient to treat an osteochondral defect therein. The joint may be a knee joint, a shoulder joint, a hip joint, an ankle joint, a first metatarsal-phalangeal joint, or the like. In exemplary embodiments, the joint is a knee joint, the first implant is a femoral implant, and the second implant is a tibial implant.

### CROSS-REFERENCES TO RELATED APPLICATIONS

This application claims the benefit of U.S. Provisional Patent Application No. 63/146,491 filed Feb. 5, 2021, the disclosure of which is incorporated herein by reference.

### BACKGROUND OF THE INVENTION

Embodiments of the present invention relate generally to arthroplasty systems and methods, and in particular encompass minimal resurfacing unicompartmental knee resurfacing implant systems and methods for osteochondral defects.

### BRIEF SUMMARY OF THE INVENTION

Unicompartmental joint resurfacing implants for osteochondral defects of the articulating joint surfaces are disclosed here. A reduced implant footprint allows for minimal resection of the articulating joint surfaces. Porous in-growth and barbed peg features allow for strong, cement-less fixation. A prosthetic for a concave joint surface can be implanted with an angled access to the joint.

In a first aspect, embodiments of the present invention encompass unicompartmental knee arthroplasty resurfacing system and methods for their use and manufacture. An exemplary unicompartmental knee arthroplasty resurfacing system can include a femoral implant and a tibial implant. The femoral implant can include a convex bearing surface portion and a subchondral surface portion. The tibial implant can include a concave bearing surface portion and a subchondral surface portion. In some cases, the concave bearing surface portion of the tibial implant has a rim that defines a proximal plane, the subchondral surface portion of the tibial implant defines a distal plane, and the proximal plane defined by the rim of the concave bearing surface portion of the tibial implant is non-parallel to the distal plane defined by the subchondral surface portion of the tibial implant. In some cases, the concave bearing surface portion of the tibial implant has a rim that defines a proximal plane, the subchondral surface portion of the tibial implant defines a distal plane, and the proximal plane defined by the rim of the concave bearing surface portion of the tibial implant is parallel to the distal plane defined by the subchondral surface portion of the tibial implant. In some cases, the femoral implant further includes at least one proximal peg. In some cases, a proximal peg of the femoral implant includes at least one barb. In some cases, the femoral implant further includes at least one anti-rotation spike. In some cases, the tibial implant further includes at least one distal peg. In some cases, a distal peg of the tibial implant includes at least one barb. In some cases, the concave bearing surface portion of the tibial implant has a rim that defines a proximal plane, the subchondral surface portion of the tibial implant defines a distal plane, the tibial implant further includes at least one distal peg, and a distal peg of the tibial implant defines an axis that is perpendicular to the distal plane defined by the subchondral surface portion of the tibial implant and that is non perpendicular to the proximal plane defined by the rim of the concave bearing surface portion of the tibial implant. In some cases, the tibial implant further includes at least one anti-rotation spike. In some cases, the convex bearing surface portion of the femoral implant includes nonporous titanium. In some cases, the subchondral surface portion of the femoral implant includes porous titanium. In some cases, a peg of the femoral implant includes multiple barbs. In some cases, the femoral implant further includes one or more anti-rotation spikes. In some cases, the femoral implant further includes one or more proximal pegs. In some cases, the concave bearing surface portion of the tibial implant includes ultra high molecular weight polyethylene. In some cases, the subchondral surface portion of the tibial implant has a proximal side and a distal side, and the concave bearing surface portion of the tibial implant is compression molded with the proximal side of the subchondral surface portion of the tibial implant. In some cases, the proximal side of the subchondral surface portion of the tibial implant includes porous titanium. In some cases, the distal side of the subchondral surface portion of the tibial implant includes an irregular lattice. In some cases, a distal peg of the tibial implant includes one or more barbs. In some cases, the tibial implant further includes one or more anti-rotation spikes. In some cases, the tibial implant further includes one or more distal pegs. In some cases, the convex bearing surface portion of the femoral implant includes a round profile. In some cases, the convex bearing surface portion of the femoral implant includes an oblong racetrack profile. In some cases, the convex bearing surface portion of the femoral implant includes a three-circle profile. In some cases, a proximal peg of the femoral implant includes a trabecular porous structure. In some cases, a distal peg of the tibial implant includes a trabecular porous structure. In some cases, the convex bearing surface portion of the femoral implant includes nonporous stainless steel, nonporous cobalt chrome, nonporous ceramic, or any combination thereof. In some cases, the proximal side of the subchondral surface portion of the tibial implant includes nonporous stainless steel, nonporous cobalt chrome, nonporous ceramic, or any combination thereof. In some cases, the convex bearing surface portion of the femoral implant includes a round profile having a diameter value within a range from about 12 mm to about 20 mm. In some cases, the convex bearing surface portion of the femoral implant includes an oblong racetrack profile having a length value within a range from about 20 mm to about 35 mm. In some cases, the convex bearing surface portion of the femoral implant includes an oblong racetrack profile having a width value within a range from about 12 mm to about 20 mm. In some cases, the femoral implant has a thickness value within a range from about 5 mm to about 10 mm. In some cases, the tibial implant has a diameter value within a range from about 15 mm to about 25 mm. In some cases, the tibial implant has a thickness value that is equal to or greater than about 6.5 mm. In some cases, the femoral implant is a monolithic unit. In some cases, the tibial implant is a monolithic unit. In some cases, the femoral implant has a circular shape and further includes a bone screw fixation mechanism. In some cases, the tibial implant has a circular shape and further includes a bone screw fixation mechanism.

In another aspect, embodiments of the present invention encompass arthroplasty resurfacing systems and methods for their use and manufacture. An exemplary arthroplasty resurfacing system can include a first implant and a second implant. The first implant can have a convex bearing surface portion and a subchondral surface portion. The second implant can have a concave bearing surface portion and a subchondral surface portion. The system can be configured for implantation into a joint of a patient. In some cases, the joint is a knee joint, a shoulder joint, a hip joint, an ankle joint, or a first metatarsal-phalangeal joint. In some cases, the first implant includes a femoral implant, the second implant includes a tibial implant, and the joint is a knee joint. In some cases, the concave bearing surface portion of the second implant has a rim that defines a proximal plane, the subchondral surface portion of the second implant defines a distal plane, and the proximal plane defined by the rim of the concave bearing surface portion of the second implant is non-parallel to the distal plane defined by the subchondral surface portion of the second implant. In some cases, the concave bearing surface portion of the second implant has a rim that defines a proximal plane, the subchondral surface portion of the second implant defines a distal plane, and the proximal plane defined by the rim of the concave bearing surface portion of the second implant is parallel to the distal plane defined by the subchondral surface portion of the second implant. In some cases, the first implant further includes at least one proximal peg. In some cases, a proximal peg of the first implant includes at least one barb. In some cases, the first implant further includes at least one anti-rotation spike. In some cases, the second implant further includes at least one distal peg. In some cases, a distal peg of the second implant includes at least one barb. In some cases, the concave bearing surface portion of the second implant has a rim that defines a proximal plane, the subchondral surface portion of the second implant defines a distal plane, the second implant further includes at least one distal peg, and a distal peg of the second implant defines an axis that is perpendicular to the distal plane defined by the subchondral surface portion of the second implant and that is non perpendicular to the proximal plane defined by the rim of the concave bearing surface portion of the second implant. In some cases, the second implant further includes at least one anti-rotation spike. In some cases, the convex bearing surface portion of the first implant includes nonporous titanium. In some cases, the subchondral surface portion of the first implant includes porous titanium. In some cases, a peg of the first implant includes one or more barbs. In some cases, the first implant further includes one or more anti-rotation spikes. In some cases, the first implant further includes one or more proximal pegs. In some cases, the concave bearing surface portion of the second implant includes ultra high molecular weight polyethylene. In some cases, the subchondral surface portion of the second implant has a proximal side and a distal side, and the concave bearing surface portion of the second implant is compression molded with the proximal side of the subchondral surface portion of the second implant. In some cases, the proximal side of the subchondral surface portion of the second implant includes porous titanium. In some cases, the distal side of the subchondral surface portion of the second implant includes an irregular lattice. In some cases, a distal peg of the second implant includes one or more barbs. In some cases, the second implant further includes one or more anti-rotation spikes. In some cases, the second implant further includes one or more distal pegs. In some cases, the convex bearing surface portion of the first implant includes a round profile. In some cases, the convex bearing surface portion of the first implant includes an oblong racetrack profile. In some cases, the convex bearing surface portion of the first implant includes a three-circle profile. In some cases, a proximal peg of the first implant includes a trabecular porous structure. In some cases, a distal peg of the second implant includes a trabecular porous structure. In some cases, the convex bearing surface portion of the first implant includes nonporous stainless steel, nonporous cobalt chrome, nonporous ceramic, or any combination thereof. In some cases, the proximal side of the subchondral surface portion of the second implant includes nonporous stainless steel, nonporous cobalt chrome, nonporous ceramic, or any combination thereof. In some cases, the convex bearing surface portion of the first implant includes a round profile having a diameter value within a range from about 12 mm to about 20 mm. In some cases, the convex bearing surface portion of the first implant includes an oblong racetrack profile having a length value within a range from about 20 mm to about 35 mm. In some cases, the convex bearing surface portion of the first implant includes an oblong racetrack profile having a width value within a range from about 12 mm to about 20 mm. In some cases, the first implant has a thickness value within a range from about 5 mm to about 10 mm. In some cases, the second implant has a diameter value within a range from about 15 mm to about 25 mm. In some cases, the second implant has a thickness value that is equal to or greater than about 6.5 mm. In some cases, the first implant is a monolithic unit. In some cases, the second implant is a monolithic unit. In some cases, the first implant has a circular shape and further includes a bone screw fixation mechanism. In some cases, the second implant has a circular shape and further includes a bone screw fixation mechanism.

In another aspect, embodiments of the present invention encompass systems and methods for implanting an arthroplasty resurfacing system into a joint of a patient. Exemplary methods may include engaging a first implant of the resurfacing system with a distal portion of a first bone of the joint of the patient, where the first implant includes a convex bearing surface portion and a subchondral surface portion. Methods may further include engaging a second implant of the resurfacing system with a proximal portion of a second bone of the joint of the patient, where the second implant includes a concave bearing surface portion and a subchondral surface portion. In some cases, the joint is a knee joint, a shoulder joint, a hip joint, an ankle joint, or a first metatarsal-phalangeal joint. In some cases, the joint is a knee joint, the first implant includes a femoral implant, and the second implant includes a tibial implant. In some cases, the concave bearing surface portion of the second implant has a rim that defines a proximal plane, the subchondral surface portion of the second implant defines a distal plane, and the proximal plane defined by the rim of the concave bearing surface portion of the second implant is non-parallel to the distal plane defined by the subchondral surface portion of the second implant. In some cases, the concave bearing surface portion of the second implant has a rim that defines a proximal plane, the subchondral surface portion of the second implant defines a distal plane, and the proximal plane defined by the rim of the concave bearing surface portion of the second implant is parallel to the distal plane defined by the subchondral surface portion of the second implant. In some cases, the first implant further includes at least one proximal peg. In some cases, a proximal peg of the first implant includes at least one barb. In some cases, the first implant further includes at least one anti-rotation spike. In some cases, the second implant further includes at least one distal peg. In some cases, a distal peg of the second implant includes at least one barb. In some cases, the concave bearing surface portion of the second implant has a rim that defines a proximal plane, the subchondral surface portion of the second implant defines a distal plane, the second implant further includes at least one distal peg, and a distal peg of the second implant defines an axis that is perpendicular to the distal plane defined by the subchondral surface portion of the second implant and that is non perpendicular to the proximal plane defined by the rim of the concave bearing surface portion of the second implant. In some cases, the second implant further includes at least one anti-rotation spike. In some cases, the convex bearing surface portion of the first implant includes nonporous titanium. In some cases, the subchondral surface portion of the first implant includes porous titanium. In some cases, a peg of the first implant includes one or more barbs. In some cases, the first implant further includes one or more anti-rotation spikes. In some cases, the first implant further includes one or more proximal pegs. In some cases, the concave bearing surface portion of the second implant includes ultra high molecular weight polyethylene. In some cases, the subchondral surface portion of the second implant has a proximal side and a distal side, and the concave bearing surface portion of the second implant is compression molded with the proximal side of the subchondral surface portion of the second implant. In some cases, the proximal side of the subchondral surface portion of the second implant includes porous titanium. In some cases, the distal side of the subchondral surface portion of the second implant includes an irregular lattice. In some cases, the distal peg of the second implant includes one or more barbs. In some cases, the second implant further includes one or more anti-rotation spikes. In some cases, the second implant further includes one or more distal pegs. In some cases, the convex bearing surface portion of the first implant includes a round profile. In some cases, the convex bearing surface portion of the first implant includes an oblong racetrack profile. In some cases, the convex bearing surface portion of the first implant includes a three-circle profile. In some cases, a proximal peg of the first implant includes a trabecular porous structure. In some cases, a distal peg of the second implant includes a trabecular porous structure. In some cases, the convex bearing surface portion of the first implant includes nonporous stainless steel, nonporous cobalt chrome, nonporous ceramic, or any combination thereof. In some cases, the proximal side of the subchondral surface portion of the second implant includes nonporous stainless steel, nonporous cobalt chrome, nonporous ceramic, or any combination thereof. In some cases, the convex bearing surface portion of the first implant includes a round profile having a diameter value within a range from about 12 mm to about 20 mm. In some cases, the convex bearing surface portion of the first implant includes an oblong racetrack profile having a length value within a range from about 20 mm to about 35 mm. In some cases, the convex bearing surface portion of the first implant includes an oblong racetrack profile having a width value within a range from about 12 mm to about 20 mm. In some cases, the first implant has a thickness value within a range from about 5 mm to about 10 mm. In some cases, the second implant has a diameter value within a range from about 15 mm to about 25 mm. In some cases, the second implant has a thickness value that is equal to or greater than about 6.5 mm. In some cases, the first implant is a monolithic unit. In some cases, the second implant is a monolithic unit. In some cases, the first implant has a circular shape and further includes a bone screw fixation mechanism. In some cases, the second implant has a circular shape and further includes a bone screw fixation mechanism.

These and other embodiments are described in further detail in the following description related to the appended drawing figures.

### BRIEF DESCRIPTION OF THE DRAWINGS

Specific embodiments of the disclosed device, delivery systems, or methods will now be described with reference to the drawings. Nothing in this detailed description is intended to imply that any particular component, feature, or step is essential to the invention.

FIGS. 1A to 1C depict aspects of arthroplasty system implants, in accordance with some embodiments.

FIG. 1D illustrates aspects of arthroplasty method, in accordance with some embodiments.

FIGS. 2A and 2B illustrate aspects of a first implant of an arthroplasty system, in accordance with some embodiments.

FIG. 3 illustrates aspects of a first implant of an arthroplasty system, in accordance with some embodiments.

FIGS. 4A to 4G illustrate aspects of first implants of arthroplasty systems, in accordance with some embodiments.

FIGS. 5A and 5B illustrate aspects of a first implant of an arthroplasty system, in accordance with some embodiments.

FIG. 6 illustrates aspects of a first implant of an arthroplasty system, in accordance with some embodiments.

FIGS. 7A to 7F illustrate aspects of first implants of arthroplasty systems, in accordance with some embodiments.

FIG. 8 illustrates aspects of a first implant of an arthroplasty system, in accordance with some embodiments.

FIGS. 9A and 9B illustrate aspects of a first implant of an arthroplasty system, in accordance with some embodiments.

FIGS. 10A and 10B illustrate aspects of a first implant of an arthroplasty system, in accordance with some embodiments.

FIGS. 11A and 11B illustrate aspects of a second implant of an arthroplasty system, in accordance with some embodiments.

FIG. 12 illustrates aspects of a second implant of an arthroplasty system, in accordance with some embodiments.

FIG. 13 illustrates aspects of a second implant of an arthroplasty system, in accordance with some embodiments.

FIG. 14 illustrates aspects of a second implant of an arthroplasty system, in accordance with some embodiments.

FIGS. 15A to 15C illustrate aspects of a second implant of an arthroplasty system, in accordance with some embodiments.

FIGS. 16A and 16B illustrate aspects of a second implant of an arthroplasty system, in accordance with some embodiments.

FIG. 17 illustrates aspects of a second implant of an arthroplasty system, in accordance with some embodiments.

FIG. 18 illustrates aspects of a second implant of an arthroplasty system, in accordance with some embodiments.

FIG. 19 illustrates aspects of a second implant of an arthroplasty system, in accordance with some embodiments.

FIG. 20 illustrates aspects of a second implant of an arthroplasty system, in accordance with some embodiments.

FIG. 21 illustrates aspects of a second implant of an arthroplasty system, in accordance with some embodiments.

FIG. 22 illustrates aspects of a second implant of an arthroplasty system, in accordance with some embodiments.

FIGS. 23A and 23B illustrate aspects of an insertion device for an arthroplasty system, in accordance with some embodiments.

FIGS. 24A and 24B illustrate aspects of an insertion device for an arthroplasty system, in accordance with some embodiments.

FIGS. 25A to 25C illustrate aspects of an insertion device for an arthroplasty system, in accordance with some embodiments.

FIGS. 26A to 26C illustrate aspects of an insertion device for an arthroplasty system, in accordance with some embodiments.

FIGS. 27A to 27C illustrate aspects of an insertion device for an arthroplasty system, in accordance with some embodiments.

FIGS. 28A and 28B illustrate aspects of an insertion device for an arthroplasty system, in accordance with some embodiments.

FIG. 29 illustrates aspects of an insertion device for an arthroplasty system, in accordance with some embodiments.

FIGS. 30A and 30B illustrate aspects of an insertion device for an arthroplasty system, in accordance with some embodiments.

FIGS. 31A and 31B illustrate aspects of an insertion device for an arthroplasty system, in accordance with some embodiments.

FIGS. 32A and 32B illustrate aspects of an insertion device for an arthroplasty system, in accordance with some embodiments.

FIGS. 33A and 33B illustrate aspects of an insertion device for an arthroplasty system, in accordance with some embodiments.

FIGS. 34A to 34C depict aspects of an exemplary tibial implant, in accordance with some embodiments.

FIGS. 35A to 35C depict aspects of an exemplary femoral oblong implant, in accordance with some embodiments.

FIGS. 36A and 36B depict aspects of an exemplary femoral round implant, in accordance with some embodiments.

FIGS. 37A to 37D depict aspects of an exemplary tibial implant, in accordance with some embodiments.

FIGS. 38A to 38D depict aspects of an exemplary femoral implant, in accordance with some embodiments.

FIGS. 39A to 39E depict aspects of an exemplary femoral implant, in accordance with some embodiments.

FIGS. 40A to 40E depict aspects of an exemplary femoral implant, in accordance with some embodiments.

FIGS. 41A to 41E depict aspects of an exemplary femoral implant, in accordance with some embodiments.

FIGS. 42A to 42E depict aspects of an exemplary tibial implant, in accordance with some embodiments.

FIGS. 43A to 43E illustrate aspects of an insertion device for an arthroplasty system, in accordance with some embodiments.

FIGS. 44A to 44E illustrate aspects of an insertion device for an arthroplasty system, in accordance with some embodiments.

FIGS. 45A to 45E illustrate aspects of an insertion device for an arthroplasty system, in accordance with some embodiments.

FIGS. 46A to 46E illustrate aspects of an insertion device for an arthroplasty system, in accordance with some embodiments.

FIGS. 47A to 47E illustrate aspects of an insertion device for an arthroplasty system, in accordance with some embodiments.

FIGS. 48A to 48D illustrate aspects of an insertion device for an arthroplasty system, in accordance with some embodiments.

FIGS. 49A to 49G illustrate aspects of an insertion device for an arthroplasty system and related methods of use, in accordance with some embodiments.

FIGS. 50A to 50C illustrate aspects of an impactor device for an arthroplasty system, in accordance with some embodiments.

FIGS. 51A to 51C illustrate aspects of an impactor device for an arthroplasty system, in accordance with some embodiments.

### DETAILED DESCRIPTION OF THE INVENTION

Currently available partial knee systems often involve large implants that require total resection and resurfacing of the femoral condyle and the tibial plateau. The tibial implant of such systems typically includes multiple components that require in-patient assembly. The inclusion of certain tibial anchors requires resection of an additional bone surface, the anterior tibial cortex. In some currently available partial knee systems, the femoral component includes multiple components that require in-patient assembly. Known systems often require cement for insertion and fixation, compromising the strength of fixation. Additionally, certain known femoral components are very thin. Existing tibial implants may require significant bone resection that goes through important bone surfaces, such as the anterior tibial cortex. Currently available tibial implants often require cement for fixation. Embodiments of the present invention provide unique solutions that address at least some of these limitations.

Implant embodiments disclosed herein can be used to address osteochondral defects early in their development, and to prevent or delay the need for a total joint replacement required if the defect worsens over time. This approach offers an appropriate step between biological techniques and the total joint arthroplasties. The fixation features of the implant allow for stronger, cementless fixation. The strong fixation and small profile provide a high level of mobility to patients, where other approaches and implants can limit patient mobility. The angled approach helps protect healthy articulating cartilage in the joint and provides easier access to the surgeon.

Exemplary arthroplasty resurfacing systems and methods disclosed herein encompass the use of a first implant having a convex bearing surface portion and a subchondral surface portion, and a second implant having a concave bearing surface portion and a subchondral surface portion. The implant system can be configured for implantation into a joint of a patient. The joint may be, for example, a knee joint, a shoulder joint, a hip joint, an ankle joint, a first metatarsal-phalangeal joint, or the like.

In one embodiment, a joint resurfacing system includes two main implants available for the knee joint: a round, femoral implant; an angled, tibial implant. In some embodiments, the femoral implant has a convex, titanium bearing surface. In some embodiments, the femoral implant has a titanium nitride (TiN) coating on the bearing surface. The circular implant has a single peg with barbs as well as irregular, porous titanium sections on the subchondral surfaces to promote fixation. There are anti-rotation spikes from the base of the implant to prevent rotational movement.

In some embodiments, the tibial implant has a concave, ultra high molecular weight (UHMW) polyethylene bearing surface. The tibial implant has an angled bearing surface to compensate for the angled access and angle of implantation. This angle of implantation is set early in the procedure with an angled pin guide. The poly portion is compression molded onto a titanium tray, using a regular porous structure on the tray as the binding site to the poly portion. The titanium tray then has a separate, irregular lattice comprising the deeper portion of the implant to promote bone in-growth. This lattice along with a central, barbed peg promote bone fixation. The base of the implant includes anti-rotation spikes to prevent rotational movement.

In some cases, implants may only be implanted on surfaces that contain osteochondral defects. They can either be used individually or in tandem on articulating surfaces. Optional features and alternatives to certain round implant embodiments disclosed herein include a variety of diametric sizes and an implant with an oblong profile, with variable length and width. The oblong shaped implants may also vary in the number of fixation pegs. In some cases, an implant may have a different profile, such as a racetrack profile or a three-circle profile. In some cases, the three circles have similar diameters. In some cases, the three circles have different diameters. In some cases, a profile may include a first number of circle profiles of one diameter, and a second number of circle profiles having another diameter. Such profiles may impart additional steps to a reaming/preparation process.

Optional and alternative features for the tibial implants include a various diametric sizes and different bearing surface angles to complement different angles of access and insertion. This also includes optional tibial pin guides that vary in angle of pin entry.

Variations for exemplary system implants can include those without porous trabecular structures to help reduce implant thickness. Implant embodiments can also have variations to accommodate different bones and joints. Implant embodiments can have multi-peg or pegless versions. Multi-peg options can remove the need for anti-rotation spikes, and pegless options can remove the need for drilling preparation. Implant embodiments encompass versions having barbless pegs that are a trabecular porous structure.

Portions of an implant may include titanium, stainless steel, cobalt chrome, and the like. Any bearing surface can be made out of ceramic.

In some embodiments, femoral round implants range in dimension diametrically from 12.0-27.5 mm. Femoral oblong implants can range in length and width/diameter from 12.0 mm×20 mm to 27.5 mm×40 mm. In some embodiments, the overall thickness of femoral implants can vary from 4 mm to 10 mm. As explained elsewhere herein, the thickness of a femoral implant can refer to the distance from the inflection point on the bearing surface (maximum for the femoral implant) to the subchondral surface, where the porous section engages the bottom or surface of the reamed bone.

In some embodiments, tibial implants range in diameters from 15 mm to 25 mm. In some cases, the minimum overall thickness of a tibial implants is 5.0 mm. In some cases, a trough of a concave bearing surface to a subchondral surface is 5.0 mm or 5.05 mm. As explained elsewhere herein, the thickness of a tibial implant can refer to the distance from the inflection point on the bearing surface (minimum for the tibial implant) to the subchondral surface, where the porous section engages the bottom or surface of the reamed bone.

In some embodiments, the connection method between the poly and titanium sections of the tibial implant use compression molding over a porous section as a mechanical lock, which reduces the thickness of the connection site compared to other mechanical locks between similar components.

In some embodiments, angled insertion allows for improved access. In some cases, no cement required. Exemplary embodiments also facilitate the accuracy of hand reaming for fine tuning final implant fit. Exemplary embodiments also encompass limited instrument sets and/or the use of no cement, leading to reduced time for surgery and reduced risk of patient exposure.

Some system embodiments include one implant per surface/defect correction (e.g., tibial tray and tibial insert are one part). Over-molding the tibial insert onto the tibial tray can reduce the thickness of the implant, which in turn reduces the depth of bone resection. A defect-sized profile reduces the area of articulating surface that needs to the be resected. Embodiments also encompass a type of multi-/no-peg alternative. A no-peg embodiment can be a helpful option to those patients with previous surgeries/hardware (e.g., interference screws). Exemplary methods encompass a retrograde approach to the tibial implant and/or an anterograde approach for the femoral implant. In some embodiments, a threaded “bone screw” fixation technique can be used for circular implants. In exemplary embodiments, implants do not require assembly in the operating room (OR), and there is no need to include alternative embodiments with subcomponents.

Alternative embodiments can include slightly altered implants that are used in similar resurfacing procedures in other joints that frequently develop osteochondral defects, including, but not limited to, the shoulder, the hip, the ankle (talus), and the first metatarsal-phalangeal joint.

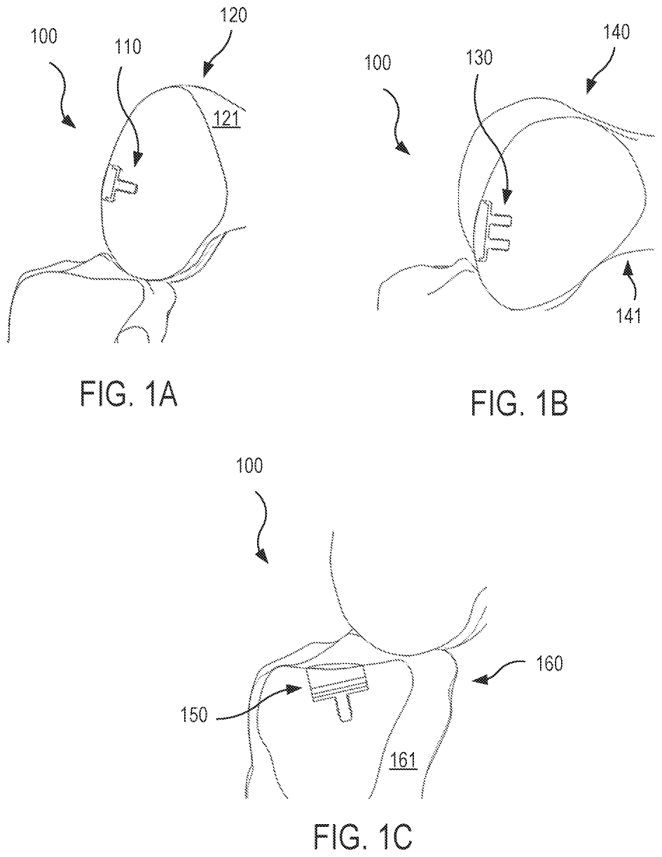

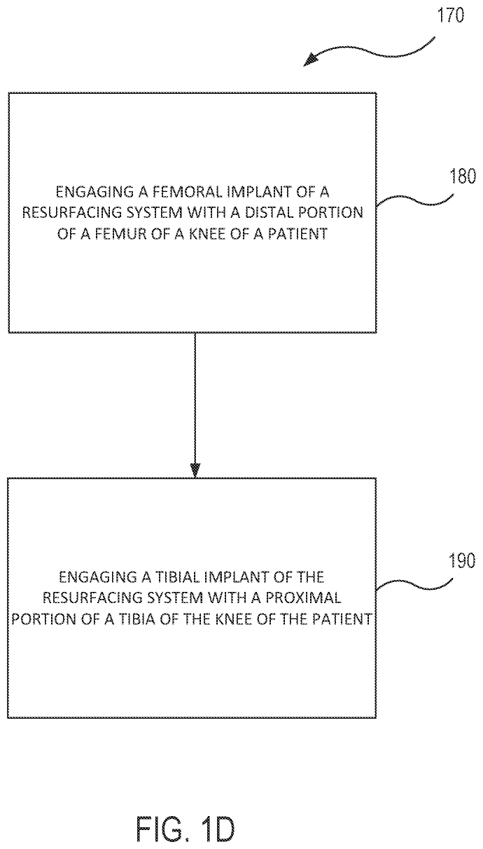

Turning now to the drawings, FIGS. 1A to 1C depict various aspects of a unicompartmental knee arthroplasty resurfacing system 100, according to embodiments of the present invention. An exemplary system may include a femoral implant and a tibial implant. For example, FIG. 1A depicts a single-peg femoral implant 110 that is engaged with a distal portion 120 of a femur 121 of a patient. Likewise, FIG. 1B depicts a multi-peg femoral implant 130 that is engaged with a distal portion 140 of a femur 141 of a patient. FIG. 1C depicts a single-peg tibial implant 150 that is engaged with a proximal portion 160 of a tibia 161 of a patient. As further discussed herein, a femoral implant can have a convex bearing surface portion, the tibial implant can have a concave bearing surface portion, and the concave and convex bearing surface portions can slidingly engage one another during use of the resurfacing system, for example as the patient's knee joint undergoes flexion and extension. FIG. 1D depicts aspects of a method 170 of implanting a unicompartmental knee arthroplasty resurfacing system into a compartment of a knee of a patient. As shown in this embodiment, the method 170 can include engaging a femoral implant of the resurfacing system with a distal portion of a femur of the knee of the patient, as indicated by step 180. The femoral implant can include a convex bearing surface portion and a subchondral surface portion. The method 170 can also include engaging a tibial implant of the resurfacing system with a proximal portion of a tibia of the knee of the patient, as indicated by step 190. The tibial implant can include a concave bearing surface portion and a subchondral surface portion.

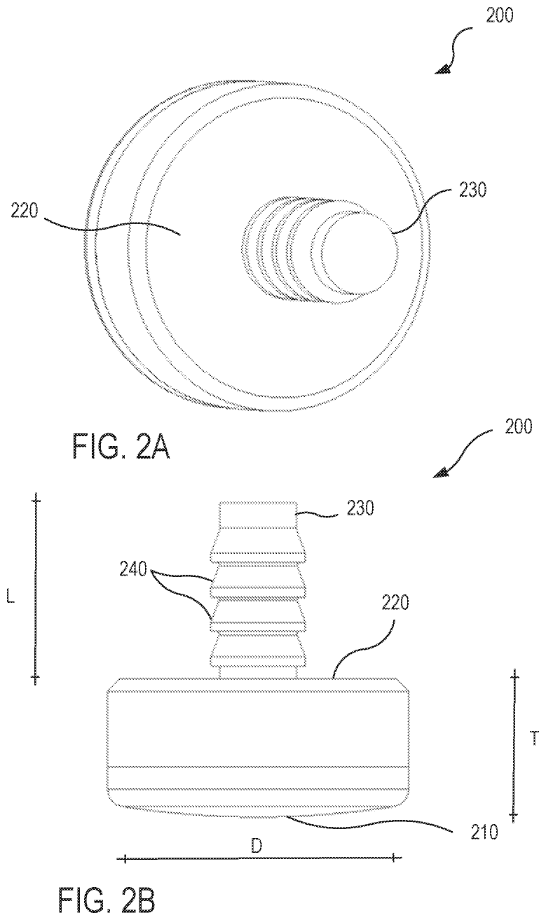

FIGS. 2A and 2B depict aspects of an exemplary femoral implant 200 according to embodiments of the present invention. As shown here, a femoral implant 200 can have a convex bearing surface portion 210 and a subchondral surface portion 220. The convex bearing surface portion 210 can operate as an articulating surface. The femoral implant 200 can also include a proximal peg 230. In the embodiment depicted here, proximal peg 230 includes multiple barbs 240. In some cases, the convex bearing surface portion 210 of the femoral implant 200 can include nonporous titanium. In some cases, the convex bearing surface portion 210 can include nonporous stainless steel, nonporous cobalt chrome, nonporous ceramic, and the like. In some cases, the subchondral surface portion 220 of the femoral implant 200 can include porous titanium. In this embodiment, the convex bearing surface portion 210 of the femoral implant 200 has a round profile. In some cases, the convex bearing surface portion 210 can provide a round profile having a diameter D with a value within a range from about 12 mm to about 20 mm. In some cases, the femoral implant 200 can have a thickness T with a value within a range from about 5 mm to about 10 mm. The thickness T of a femoral implant can refer to the distance from the inflection point on the bearing surface (maximum for the femoral implant) to the subchondral surface, where the porous section engages the bottom or surface of the reamed bone of the patient. A peg length L can have a value of about 7 mm. In some cases, the proximal peg 230 can have a trabecular porous structure. In some cases, the femoral implant 200 is a monolithic unit. In some cases, the femoral implant 200 includes a bone screw fixation mechanism.

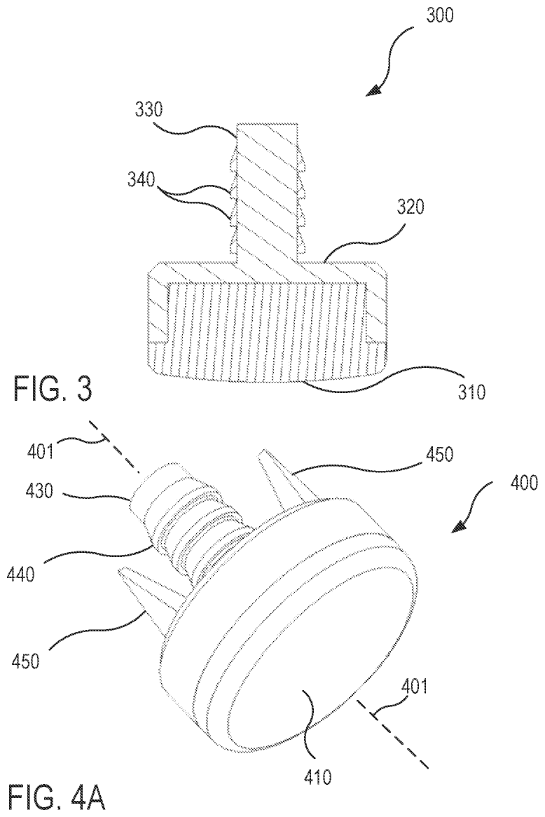

FIG. 3 depicts aspects of an exemplary femoral implant 300 according to embodiments of the present invention. As shown here, a femoral implant 300 can have a convex bearing surface portion 310 and a subchondral surface portion 320. The femoral implant 300 can also include a proximal peg 330. In the embodiment depicted here, proximal peg 330 includes multiple barbs 340. In some cases, the peg 330 and subchondral surface portion 320 include a porous material, and the barbs 340 and the convex bearing surface portion 310 includes a solid or nonporous material. In some cases, the convex bearing surface portion 310 of the femoral implant 300 can include nonporous titanium. In some cases, the convex bearing surface portion 310 can include nonporous stainless steel, nonporous cobalt chrome, nonporous ceramic, and the like. In some cases, the barbs 340 can include a nonporous or solid material. In some cases, the subchondral surface portion 320 of the femoral implant 300 can include porous titanium. In this embodiment, the convex bearing surface portion 310 of the femoral implant 300 has a round profile. In some cases, the proximal peg 330 can have a trabecular porous structure. In some cases, the femoral implant 300 is a monolithic unit.

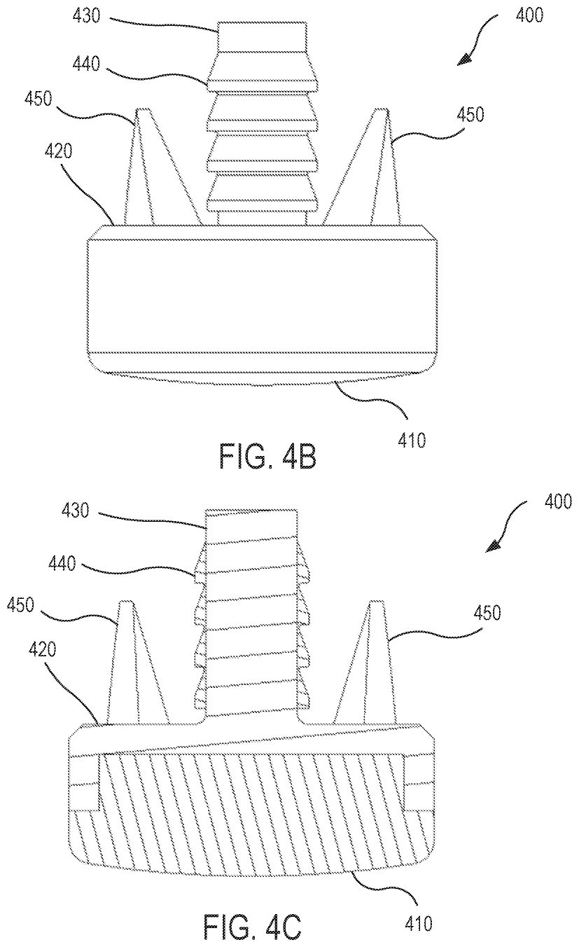

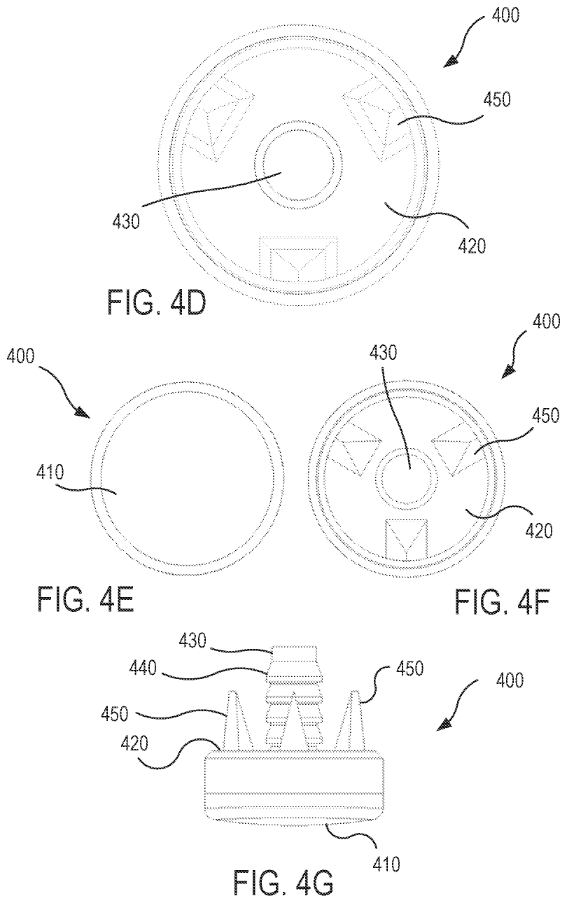

FIGS. 4A to 4G depict aspects of an exemplary femoral implant 400 according to embodiments of the present invention. As shown here, a femoral implant 400 can have a convex bearing surface portion 410 and a subchondral surface portion 420. The femoral implant 400 can also include a proximal peg 430. In the embodiment depicted here, proximal peg 430 includes multiple barbs 440. In some cases, the convex bearing surface portion 410 of the femoral implant 400 can include nonporous titanium. In some cases, the convex bearing surface portion 410 can include nonporous stainless steel, nonporous cobalt chrome, nonporous ceramic, and the like. In some cases, the femoral implant can include no porous structure. In this embodiment, the convex bearing surface portion 410 of the femoral implant 400 has a round profile. In some cases, the femoral implant 400 is a monolithic unit. In this embodiment, the femoral implant 400 includes multiple anti-rotation spikes or prongs 450. Such spikes or prongs 450 can help to prevent or inhibit the implant 400 from rotating about a central longitudinal axis 401 of the implant or proximal peg 430 when the implant is implanted in the patient's body. In some cases, the femoral implant can include no porous structure (e.g., FIG. 4B). That is, the femoral implant depicted in FIG. 4B is completely made of a solid or nonporous material.

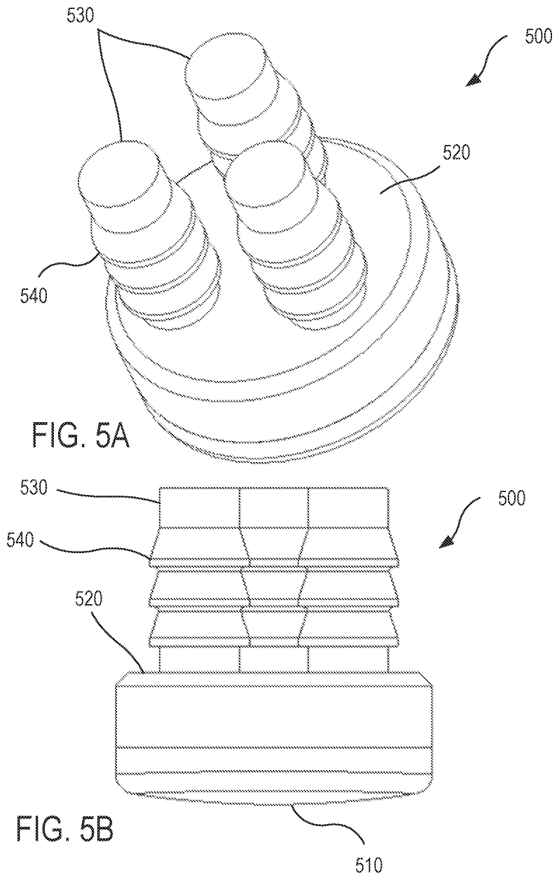

FIGS. 5A and 5B depict aspects of an exemplary femoral implant 500 according to embodiments of the present invention. As shown here, a femoral implant 500 can have a convex bearing surface portion 510 and a subchondral surface portion 520. The femoral implant 500 can also include multiple proximal pegs 530. In the embodiment depicted here, proximal pegs 530 include multiple barbs 540. In some cases, the convex bearing surface portion 510 of the femoral implant 500 can include nonporous titanium. In some cases, the convex bearing surface portion 510 can include nonporous stainless steel, nonporous cobalt chrome, nonporous ceramic, and the like. In some cases, the femoral implant can include no porous structure. In this embodiment, the convex bearing surface portion 510 of the femoral implant 500 has a round profile. In some cases, the femoral implant 500 is a monolithic unit. In this embodiment, the femoral implant 500 includes no anti-rotation spikes. The presence of multiple pegs 530 can help to prevent or inhibit the implant 500 from rotating about a central longitudinal axis thereof when implanted in the patient's body.

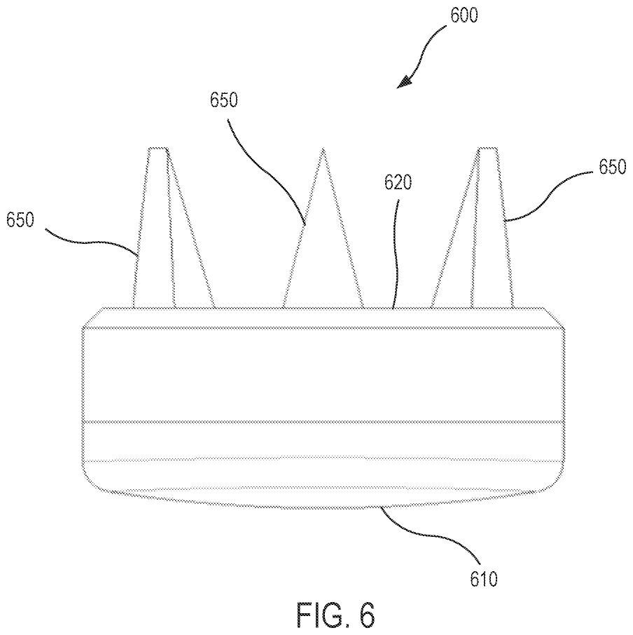

FIG. 6 depicts aspects of an exemplary femoral implant 600 according to embodiments of the present invention. As shown here, a femoral implant 600 can have a convex bearing surface portion 610 and a subchondral surface portion 620. In some cases, the convex bearing surface portion 610 of the femoral implant 600 can include nonporous titanium. In some cases, the convex bearing surface portion 610 can include nonporous stainless steel, nonporous cobalt chrome, nonporous ceramic, and the like. In some cases, the femoral implant can include no porous structure. In this embodiment, the convex bearing surface portion 610 of the femoral implant 600 has a round profile. In some cases, the femoral implant 600 is a monolithic unit. In this embodiment, the femoral implant 600 includes multiple anti-rotation spikes 650 and no proximal pegs. The presence of the spikes 650 can help to prevent or inhibit the implant 600 from rotating about a central longitudinal axis thereof when implanted in the patient's body.

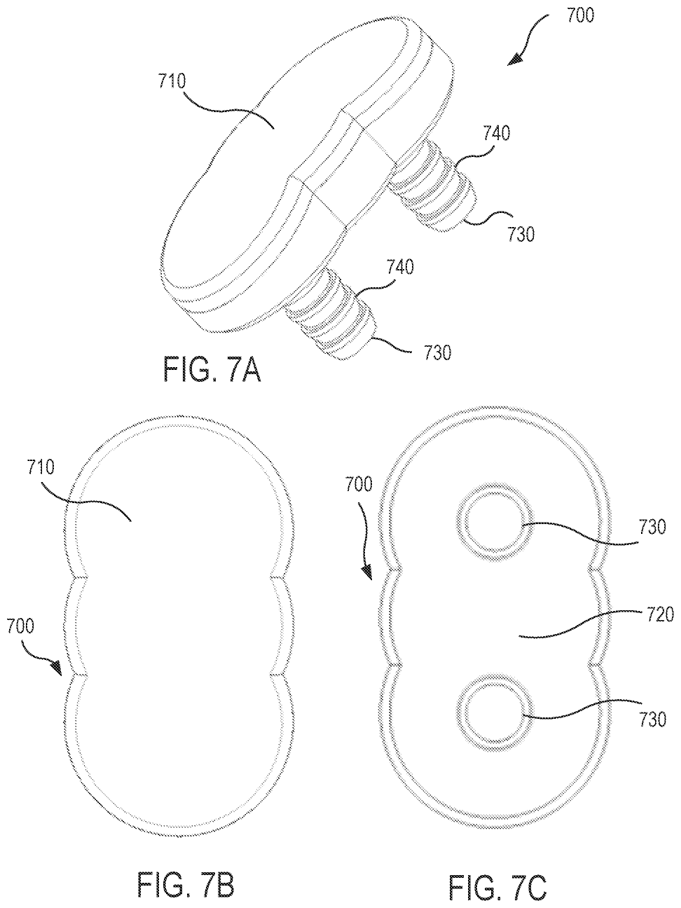

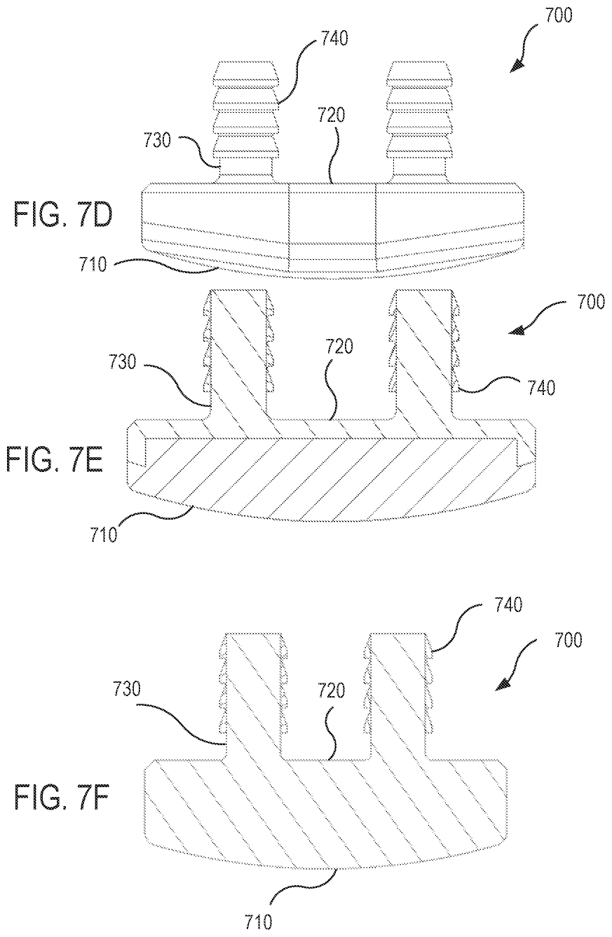

FIGS. 7A to 7F depict aspects of an exemplary femoral implant 700 according to embodiments of the present invention. As shown here, a femoral implant 700 can have a convex bearing surface portion 710 and a subchondral surface portion 720. The femoral implant 700 can also include one or more proximal pegs 730. In some cases, the subchondral surface portion 720 and/or one or more the proximal pegs 730 can include a porous material, such as porous titanium. In the embodiment depicted here, proximal pegs 730 include multiple barbs 740. In some cases, the convex bearing surface portion 710 of the femoral implant 700 can include nonporous titanium. In some cases, the convex bearing surface portion 710 can include nonporous stainless steel, nonporous cobalt chrome, nonporous ceramic, and the like. In some cases, the pegs 730 and subchondral surface portion 720 include a porous material, and the barbs 740 and the convex bearing surface portion 710 includes a solid or nonporous material (e.g., FIG. 7E). In some cases, the femoral implant can include no porous structure (e.g., FIG. 7F). That is, the femoral implant depicted in FIG. 7F is completely made of a solid or nonporous material. The convex bearing surface portion 710 of the femoral implant 700 has a three-circle or oblong profile. The three-circle profile provides a surface portion that has three sections, which allows for simple reaming with a circular reamer (e.g. in 3 adjacent reaming locations on the patient's bone, with overlapping circles). In some cases, the femoral implant 700 is a monolithic unit. In this embodiment, the femoral implant 700 includes no anti-rotation spikes, although such spikes may be present in other embodiments.

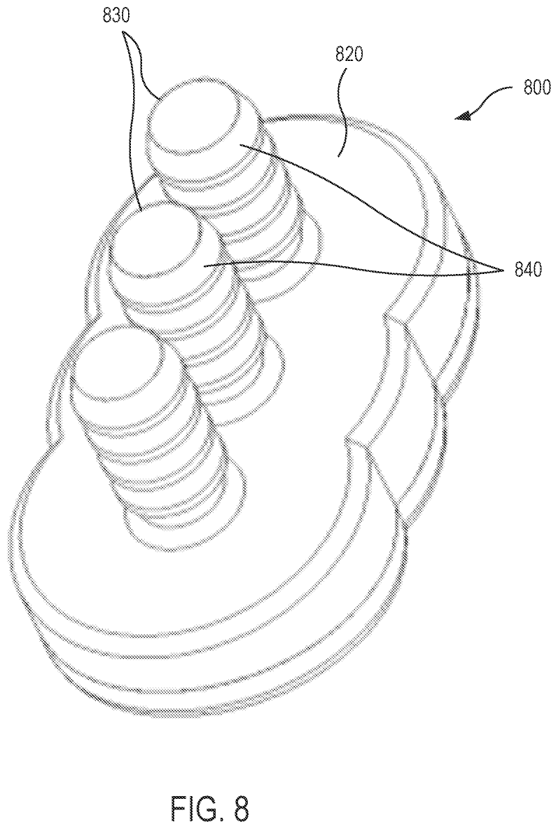

FIG. 8 depicts aspects of an exemplary femoral implant 800 according to embodiments of the present invention. As shown here, a femoral implant 800 can have a convex bearing surface portion and a subchondral surface portion 820. The femoral implant 800 can also include one or more proximal pegs 830. In some cases, the subchondral surface portion 830 and/or one or more the proximal pegs 830 can include a porous material, such as porous titanium. In the embodiment depicted here, proximal pegs 830 include multiple barbs 840. In some cases, the convex bearing surface portion of the femoral implant 800 can include nonporous titanium. In some cases, the convex bearing surface portion can include nonporous stainless steel, nonporous cobalt chrome, nonporous ceramic, and the like. In some cases, the femoral implant can include no porous structure. The femoral implant 800 has a three-circle profile. In some cases, the femoral implant 800 is a monolithic unit. In this embodiment, the femoral implant 800 includes no anti-rotation spikes, although such spikes may be present in other embodiments.

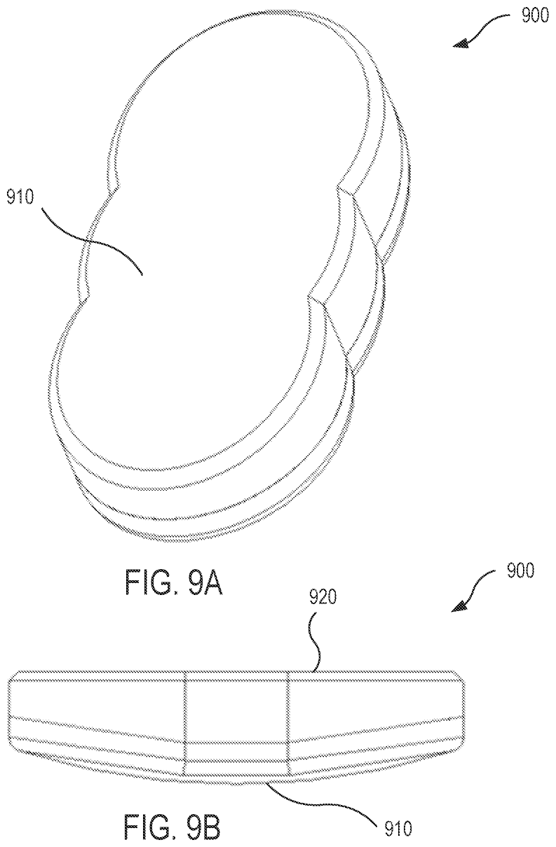

FIGS. 9A and 9B depict aspects of an exemplary femoral implant 900 according to embodiments of the present invention. As shown here, a femoral implant 900 can have a convex bearing surface portion 910 and a subchondral surface portion 920. The femoral implant 900 does not include proximal pegs, although such pegs can be present in other embodiments. In some cases, the subchondral surface portion 920 can include a porous material, such as porous titanium. In some cases, the convex bearing surface portion of the femoral implant 800 can include nonporous titanium. In some cases, the convex bearing surface portion 910 can include nonporous stainless steel, nonporous cobalt chrome, nonporous ceramic, and the like. In some cases, the femoral implant can include no porous structure. The femoral implant 900 has a three-circle profile. In some cases, the femoral implant 900 is a monolithic unit. In this embodiment, the femoral implant 900 includes no anti-rotation spikes, although such spikes may be present in other embodiments.

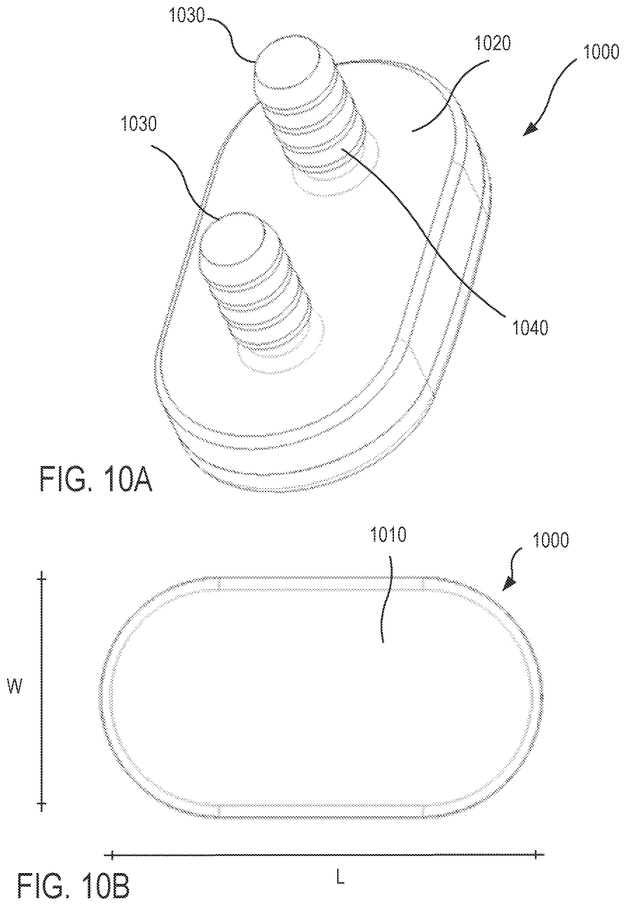

FIGS. 10A and 10B depict aspects of an exemplary femoral implant 1000 according to embodiments of the present invention. As shown here, a femoral implant 1000 can have a convex bearing surface portion 1010 and a subchondral surface portion 1020. The femoral implant 1000 can also include one or more proximal pegs 1030. In some cases, the subchondral surface portion 1020 and/or one or more the proximal pegs 1030 can include a porous material, such as porous titanium. In the embodiment depicted here, proximal pegs 1030 include multiple barbs 1040. In some cases, the convex bearing surface portion 1010 of the femoral implant 1000 can include nonporous titanium. In some cases, the convex bearing surface portion 1010 can include nonporous stainless steel, nonporous cobalt chrome, nonporous ceramic, and the like. In some cases, the femoral implant can include no porous structure. The convex bearing surface portion 1010 of the femoral implant 1000 has an oblong racetrack profile. In some cases, the oblong racetrack profile has a length L with a value within a range from about 20 mm to about 35 mm. In some cases, the oblong racetrack profile has a width W with a value within a range from about 12 mm to about 20 mm. In some cases, the femoral implant 1000 is a monolithic unit. In this embodiment, the femoral implant 1000 includes no anti-rotation spikes, although such spikes may be present in other embodiments.

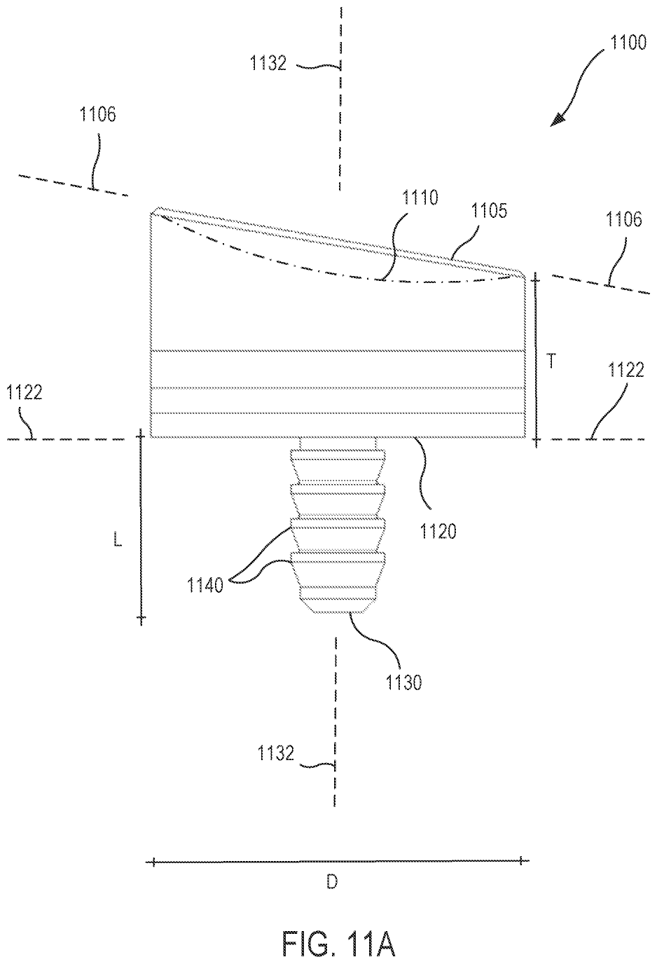

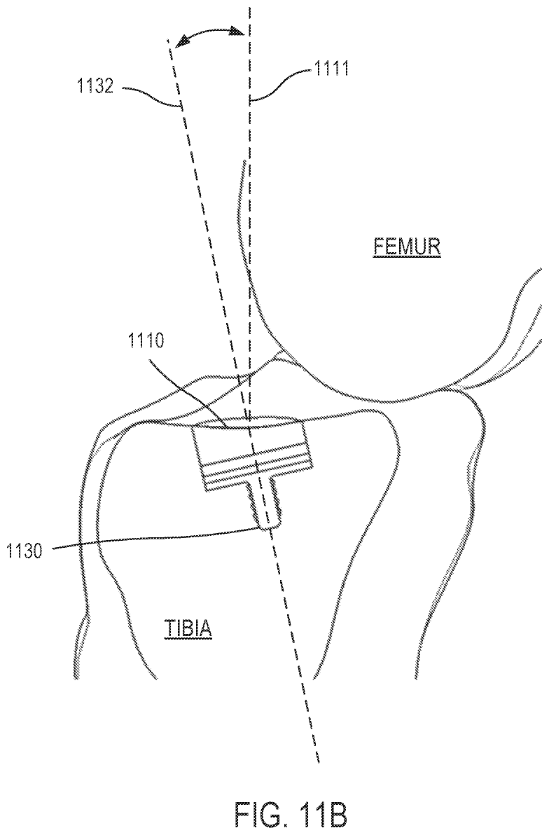

FIG. 11A depicts aspects of an exemplary tibial implant 1100 according to embodiments of the present invention. As shown here, a tibial implant 1100 can have a concave bearing surface portion 1110 (hidden from view) and a subchondral surface portion 1120. The tibial implant 1100 can also include a distal peg 1130. In the embodiment depicted here, distal peg 1130 can include one or more barbs 1140. In some cases, the concave bearing surface portion 1110 of the tibial implant 1100 can include ultra high molecular weight (UHMW) polyethylene. In some cases, the proximal side of the subchondral surface portion 1120 of the tibial implant 1100 includes porous titanium. In some cases, the proximal side of the subchondral surface portion 1120 of the tibial implant 1100 includes nonporous stainless steel, nonporous cobalt chrome, and/or nonporous ceramic. In some cases, the distal side of the subchondral surface portion 1120 of the tibial implant 1100 includes an irregular lattice. In this embodiment, the concave bearing surface portion 1110 of the femoral implant 1100 has a round profile. In some cases, the concave bearing surface portion 1110 can provide a round profile having a diameter D with a value within a range from about 15 mm to about 25 mm. In some cases, the tibial implant 1100 can have a thickness T with a value that is equal to or greater than about 6.5 mm. The thickness T of a tibial implant can refer to the distance from the inflection point on the bearing surface (minimum for the tibial implant) to the subchondral surface, where the porous section engages the bottom or surface of the reamed bone. A peg length L can have a value of about 5.5 mm. In some cases, the distal peg 1130 can have a trabecular porous structure. In some cases, the tibial implant 1100 is a monolithic unit. In this embodiment, the tibial implant 1100 includes no anti-rotation spikes, although such spikes may be present in other embodiments. In some cases, the tibial implant 1100 includes a bone screw fixation mechanism.

As shown in FIG. 11A, the concave bearing surface portion 1110 of the tibial implant 1100 can have a rim 1105 that defines a proximal plane 1106, and the subchondral surface portion 1120 of the tibial implant 1100 can define distal plane 1122. In some embodiments, the proximal plane 1106 defined by the rim 1105 of the concave bearing surface portion 1110 of the tibial implant 1100 is non-parallel to the distal plane 1122 defined by the subchondral surface portion 1120 of the tibial implant 1100. The distal peg 1130 can define a longitudinal axis 1132. In some cases, the longitudinal axis 1132 defined by the distal peg 1130 can be perpendicular to the distal plane 1122 defined by the subchondral surface portion 1120 of the tibial implant 1100 and non-perpendicular to the proximal plane 1106 defined by the rim 1105 of the concave bearing surface portion 1110 of the tibial implant 1100. As shown in FIG. 11B, upon implantation, the longitudinal axis 1132 defined by the distal peg 1130 is at an angle A from an axis 1111 that is normal to the concave bearing surface portion 1110. In some cases, angle A has a value of about 10 degrees. As shown here, the bottom or distal surface of the tibial tray can be perpendicular to the peg.

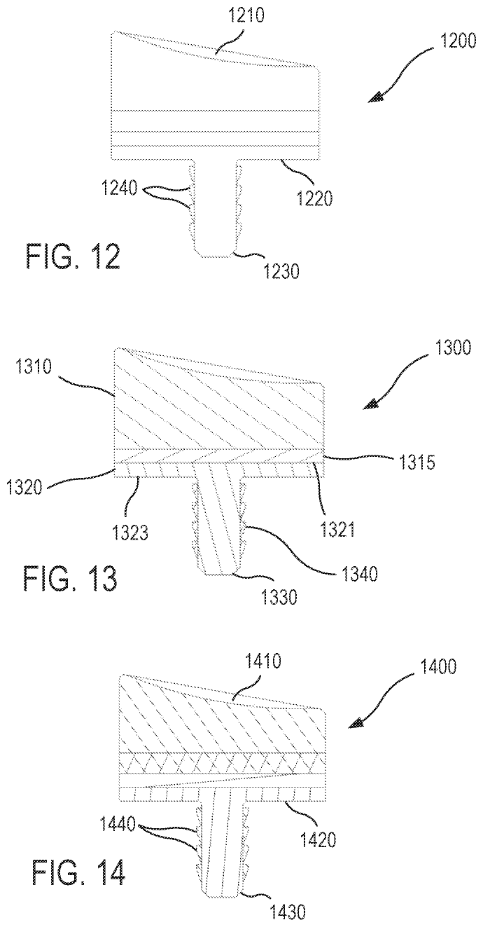

FIG. 12 depict aspects of an exemplary tibial implant 1200 according to embodiments of the present invention. Tibial implant 1200 includes a concave bearing surface 1210, a subchondral surface 1220, and a distal peg 1230 having multiple barbs 1240.

FIG. 13 depict aspects of an exemplary tibial implant 1300 according to embodiments of the present invention. In the cross-section view provided here, the implant 1300 includes a concave bearing surface portion 1310 (which may include a nonporous polymeric material), a subchondral surface portion 1320 (which may include a porous metal material) having a proximal side 1321 and a distal side 1323, and multiple barbs 1340 (which may include a nonporous metal material) on a distal peg 1330 (which may include a porous metal material). The concave bearing surface portion 1310 of the tibial implant can be compression molded with the proximal side 1321 of the subchondral surface portion 1320 of the tibial implant 1300. In some cases, due to the compression molding process, there may be an overlap area 1315. In some cases, the concave bearing surface portion 1310 includes ultra high molecular weight (UHMW) polyethylene, the subchondral surface portion 1320 and distal peg 1330 include a porous metal material, and the barbs 1340 include a solid metal material. The overlap area or portion 1315 can include both UHMW polyethylene and porous metal, and can be present as a result of compression molding. For instance, the concave bearing surface portion 1310 of the tibial implant can be compression molded with the proximal side 1321 of the subchondral surface portion of the tibial implant.

FIG. 14 depict aspects of an exemplary tibial implant 1400 according to embodiments of the present invention. In the cross-section view provided here, it can be seen that tibial implant 1400 includes a concave bearing surface 1410, a subchondral surface 1420, and a distal peg 1430 having multiple barbs 1440.

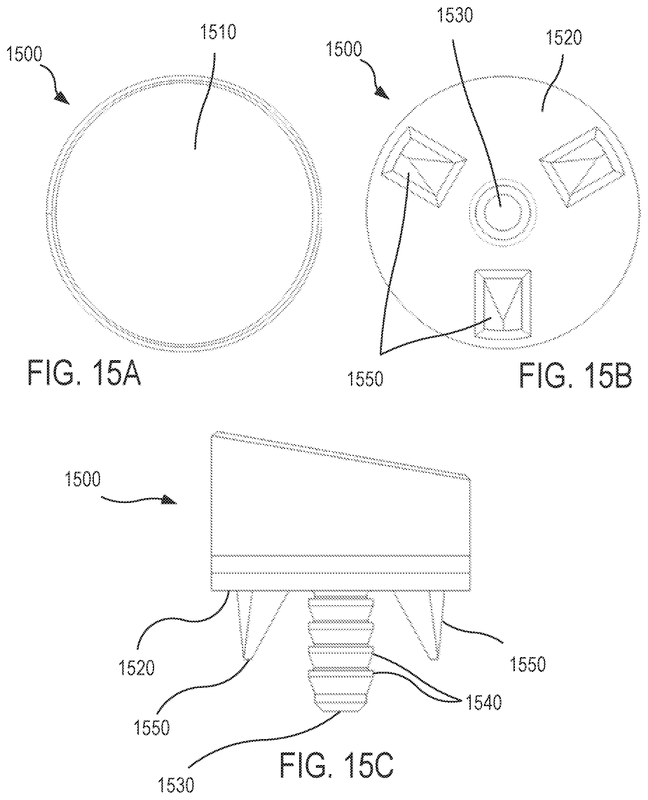

FIGS. 15A to 15C depict aspects of an exemplary tibial implant 1500 according to embodiments of the present invention. As shown here, a tibial implant 1500 can have a concave bearing surface portion 1510 and a subchondral surface portion 1520. The tibial implant 1500 can also include a distal peg 1530. In the embodiment depicted here, distal peg 1530 can include one or more barbs 1540. In some cases, the concave bearing surface portion 1510 of the tibial implant 1500 can include ultra high molecular weight (UHMW) polyethylene. In some cases, the proximal side of the subchondral surface portion 1530 of the tibial implant 1500 includes porous titanium. In some cases, the proximal side of the subchondral surface portion 1520 of the tibial implant 1500 includes nonporous stainless steel, nonporous cobalt chrome, and/or nonporous ceramic. In some cases, the distal side of the subchondral surface portion 1520 of the tibial implant 1500 includes an irregular lattice. In this embodiment, the concave bearing surface portion 1510 of the femoral implant 1500 has a round profile. In some cases, the distal peg 1530 can have a trabecular porous structure. In some cases, the tibial implant 1500 is a monolithic unit. In some cases, the tibial implant 1500 can include one or more anti-rotation spikes 1550, although such spikes may be absent in other embodiments.

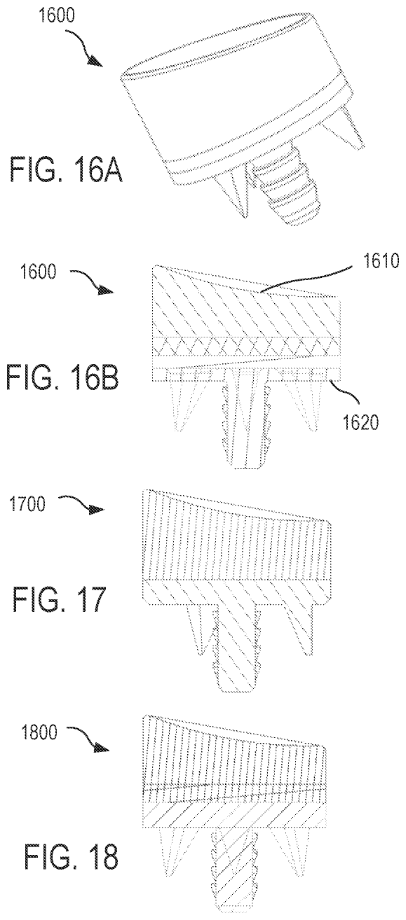

FIGS. 16A and 16B depict aspects of an exemplary tibial implant 1600 according to embodiments of the present invention. As shown here, a tibial implant 1600 can have a concave bearing surface portion 1610 and a subchondral surface portion 1620. FIG. 16B provides a cross-section view of the implant depicted in FIG. 16A.

FIG. 17 depict aspects of an exemplary tibial implant 1700 according to embodiments of the present invention.

FIG. 18 depict aspects of an exemplary tibial implant 1800 according to embodiments of the present invention. As shown here, the tibial implant 1800 includes no trabecular bottom porous structure.

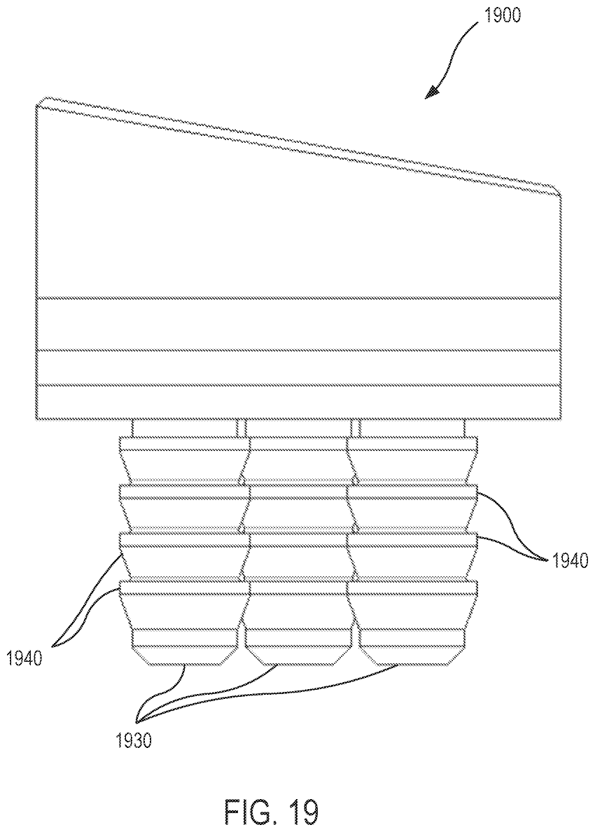

FIG. 19 depicts aspects of an exemplary tibial implant 1900 according to embodiments of the present invention. The tibial implant 1100 can include multiple distal pegs 1930. In the embodiment depicted here, distal pegs 1930 can include one or more barbs 1940.

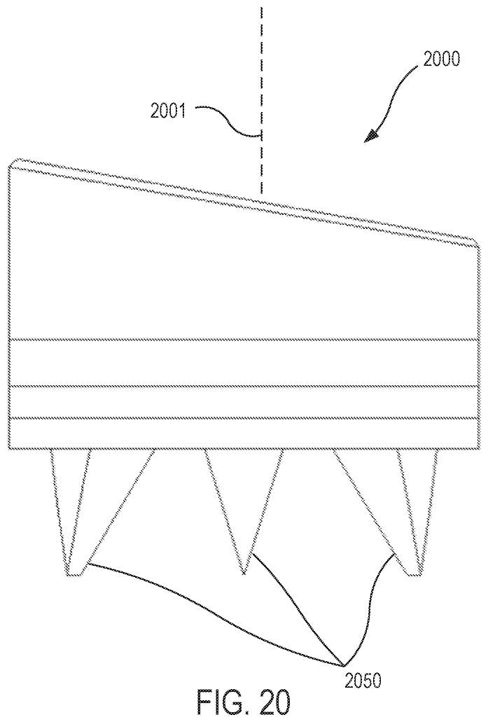

FIG. 20 depicts aspects of an exemplary tibial implant 2000 according to embodiments of the present invention. The tibial implant 2000 can include one or more anti-rotation spikes 2050. Such spikes 2050 can help to prevent or inhibit the implant 200 from rotating about a central longitudinal axis 2001 thereof when implanted in the patient's body.

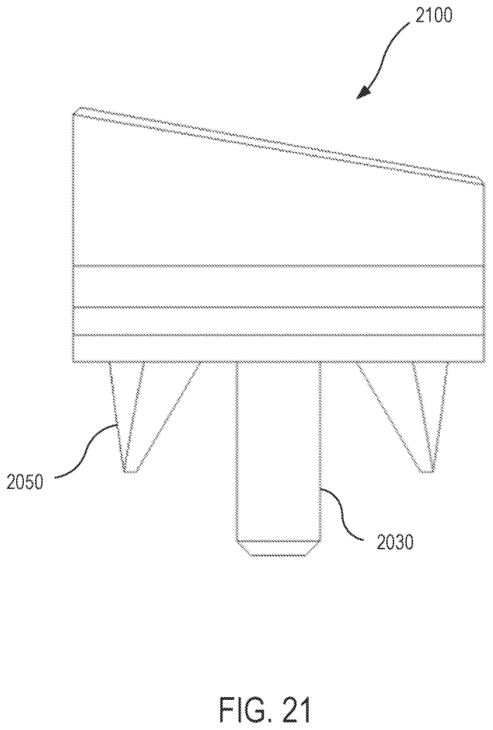

FIG. 21 depicts aspects of an exemplary tibial implant 2100 according to embodiments of the present invention. The tibial implant 2100 can include one or more anti-rotation spikes 2150. The tibial implant can also include one or more distal pegs, such as distal peg 2030. As shown here, distal peg 2030 does not have any barbs, although barbs may be present on distal pegs in other embodiments.

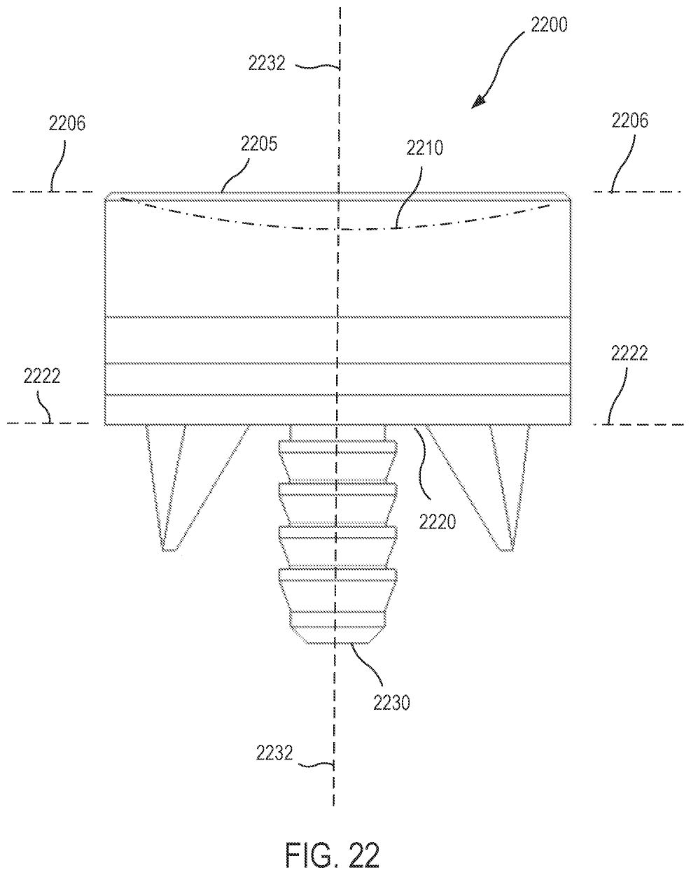

FIG. 22 depicts aspects of an exemplary tibial implant 2200 according to embodiments of the present invention. As shown here, a tibial implant 2200 can have a concave bearing surface portion 2210 (hidden from view) and a subchondral surface portion 2220. The tibial implant 2200 can also include a distal peg 2230. The concave bearing surface portion 2210 of the tibial implant 2200 can have a rim 2205 that defines a proximal plane 2206, and the subchondral surface portion 2220 of the tibial implant 2200 can define distal plane 2222. In some embodiments, the proximal plane 2206 defined by the rim 2205 of the concave bearing surface portion 2210 of the tibial implant 2200 is parallel to the distal plane 2222 defined by the subchondral surface portion 2220 of the tibial implant 2200. The distal peg 2230 can define a longitudinal axis 2232. In some cases, the longitudinal axis 2232 defined by the distal peg 2230 can be perpendicular to the distal plane 2222 defined by the subchondral surface portion 2220 of the tibial implant 2200 and also perpendicular to the proximal plane 2206 defined by the rim 2205 of the concave bearing surface portion 2210 of the tibial implant 2200. Embodiments of the present invention also encompass tibial implants where the longitudinal axis 2232 is not perpendicular to the distal plane 2222 and/or the proximal plane 2206. In some cases, tibial implant 2200 can provide a zero degree or variable angle embodiment.

Embodiments of the present invention encompass a variety of insertion devices and methods for an arthroplasty system. In some cases, insertion devices may include components such as reamers, guides, sizers, and the like. Insertion devices can be used to prepare patient tissue (e.g. bone) for receiving an implant, for positioning an implant, and/or to for inserting an implant in a patient tissue or affixing an implant to a patient tissue.

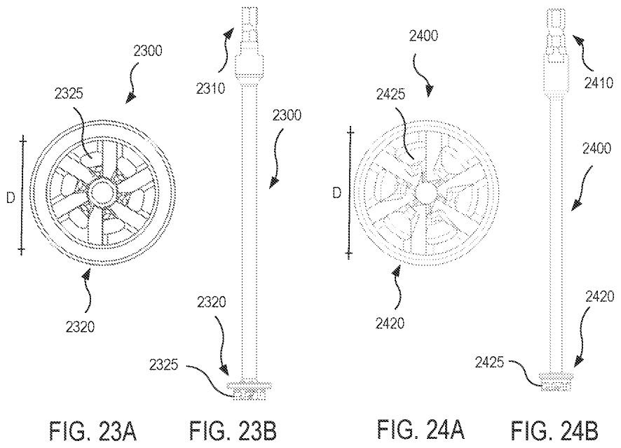

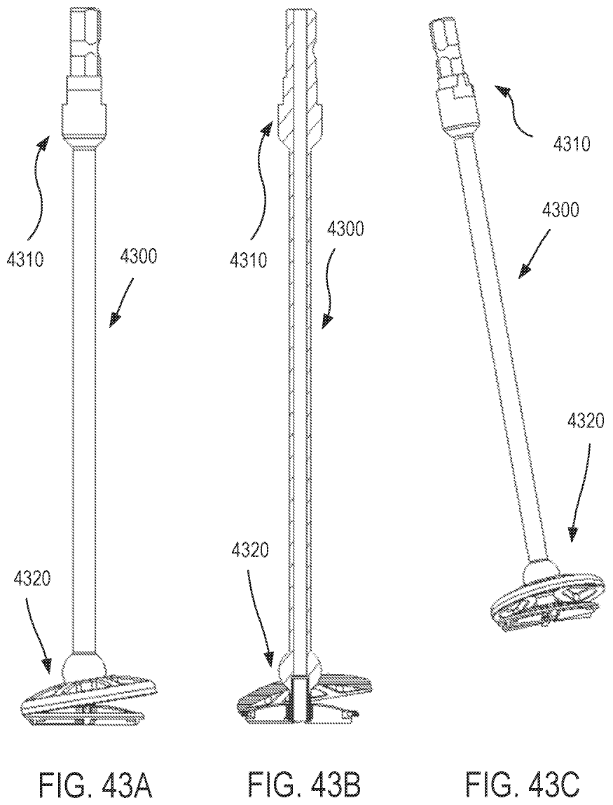

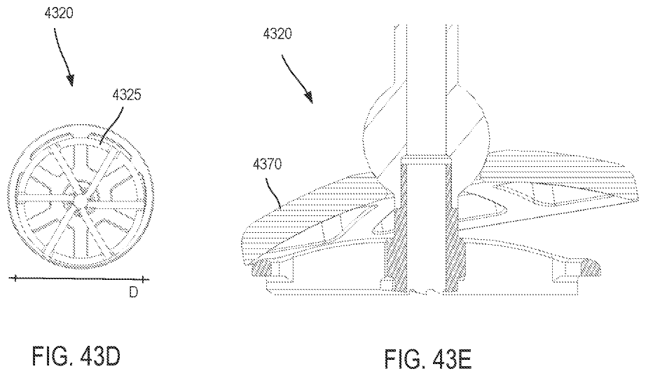

FIGS. 23A and 23B depict aspects of a femoral reamer (primary) system 2300, according to embodiments of the present invention. FIG. 23A provides a distal end view, and FIG. 23B provides a side view. The femoral reamer (primary) system 2300 includes a proximal end 2310 and a distal end 2320, where the distal end 2320 is configured to ream bone of the patient, thereby producing a recess in the bone which is sized and/or configured to receive an implant. For example, the distal end 2320 can produce a recess or hole in the distal portion of the femur. The distal end 2320 can have a drill mechanism 2325, which may include cutting or boring elements, having a diameter D, so as to produce a recess or hole of similar diameter. Related aspects of primary femoral reamer system embodiments are discussed elsewhere herein, for example in association with FIGS. 43A to 43E.

FIGS. 24A and 24B depict aspects of a femoral reamer (primary) system 2400, according to embodiments of the present invention. FIG. 24A provides a distal end view, and FIG. 24B provides a side view. The femoral reamer (primary) system 2400 includes a proximal end 2410 and a distal end 2420, where the distal end 2420 is configured to ream bone of the patient, thereby producing a recess in the bone which is sized and/or configured to receive an implant. For example, the distal end 2420 can produce a recess or hole in the distal portion of the femur. The distal end 2420 can have a drill mechanism 2425, which may include cutting or boring elements, having a diameter D, so as to produce a recess or hole of similar diameter.

According to some embodiments, FIGS. 23A and 23B depict aspects of a reamer system for a round femoral implant, and only one reamer is needed to implant a round implant. According to some embodiments, FIGS. 24A and 24B depict aspects of a primary reamer system for an oblong implant. The system of FIGS. 23A and 23B may differ from the system of FIGS. 24A and 24B in terms of ream depth, drill depth, and/or bore depth. In some cases, a larger diameter at the top of the cutting feature can operate to limit the boring depth.

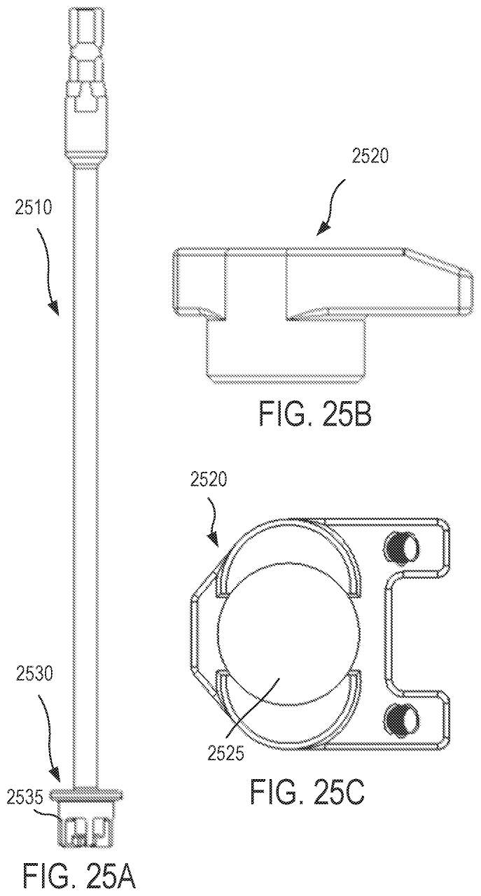

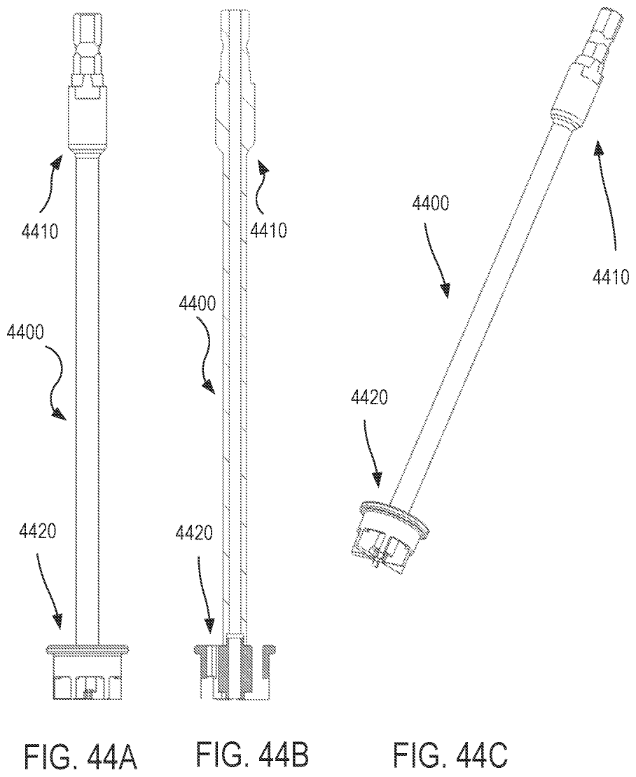

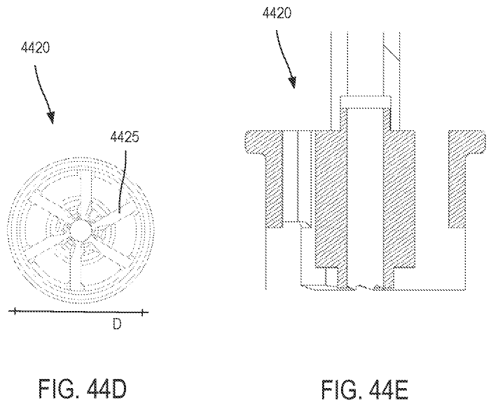

FIGS. 25A to 25C depict aspects of a femoral reamer (secondary) system, according to embodiments of the present invention. The system can include a drill assembly 2510 (as shown in FIG. 25A) and a guide 2520 (as shown in the side view of FIG. 25B and the plan view of FIG. 25C). In use, the guide 2520 can be engaged with the distal section of the patient's femur, and a drill mechanism 2535 of a distal end 2530 of the drill assembly 2510 can be aligned with the guide 2520, for example by placing the drill mechanism 2535 in an aperture 2525 of the guide 2520, so that the hole or bore in the distal femur bone produced by the reamer system is positioned as desired. Related aspects of secondary femoral reamer system embodiments are discussed elsewhere herein, for example in association with FIGS. 44A to 44E.

FIGS. 26A to 26C depicts aspects of a femoral reamer (through guide) system, according to embodiments of the present invention. The system can include a drill support 2610 (as shown in the side view of FIG. 26A and the cross-section view of FIG. 26B) and a guide 2620 (as shown in the plan view of FIG. 26C). In use, the guide 2620 can be engaged with the distal section of the patient's femur, and a distal end 2630 of the drill support 2610 can be aligned with the guide 2620, for example by aligning the distal end 2630 with an aperture 2625 of the guide 2620, and/or with lateral sections 2627, 2629 so that one or more holes or bores can be produced in the distal femur bone by a drill (not shown) that is advanced through a central aperture 2612 of the drill support 2610. Such holes or bores can then receive respective pegs of an implant. In some embodiments, the diameter of the distal end of the reamer can vary with the implant size (e.g. larger reamer distal end diameter for larger implant size, and smaller reamer distal end for smaller implant size).

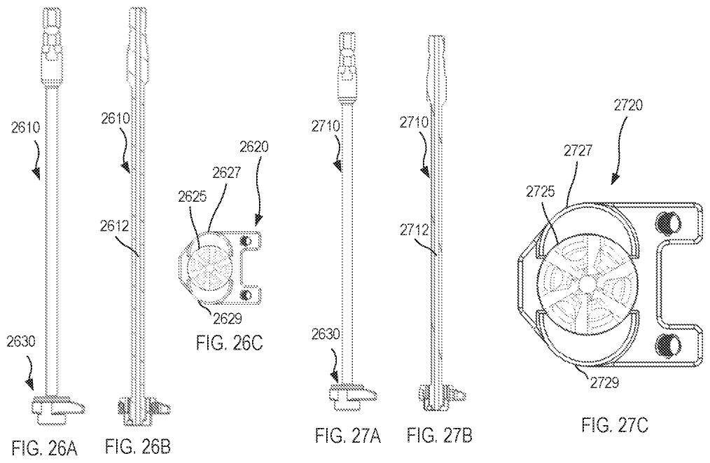

FIGS. 27A to 27C depict aspects of a femoral reamer (through guide) system, according to embodiments of the present invention. The system can include a drill support 2710 (as shown in the side view of FIG. 27A and the cross-section view of FIG. 27B) and a guide 2720 (as shown in the plan view of FIG. 27C). In use, the guide 2720 can be engaged with the distal section of the patient's femur, and a distal end 2730 of the drill support 2710 can be aligned with the guide 2720, for example by aligning the distal end 2730 with an aperture 2725 of the guide 2720, and/or with lateral sections 2727, 2729 so that one or more holes or bores can be produced in the distal femur bone by a drill (not shown) that is advanced through a central aperture 2712 of the drill support 2710. Such holes or bores can then receive respective pegs of an implant. In some embodiments, the diameter of the distal end of the reamer can vary with the implant size (e.g. larger reamer distal end diameter for larger implant size, and smaller reamer distal end for smaller implant size).

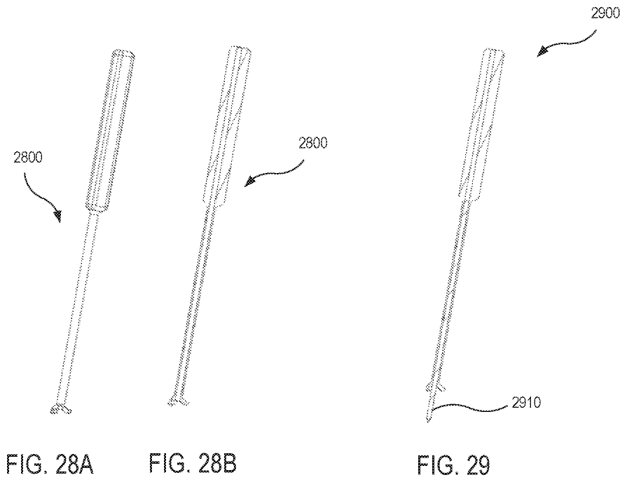

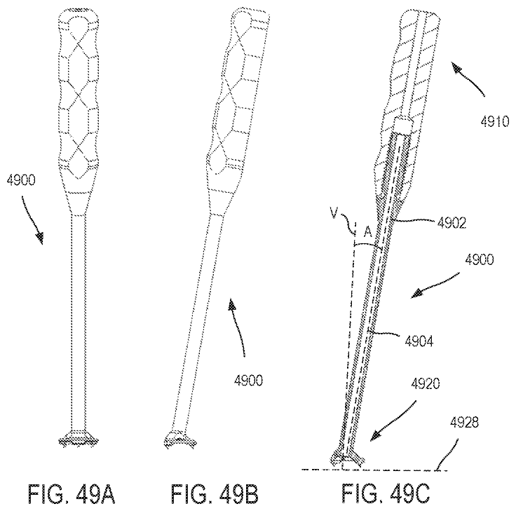

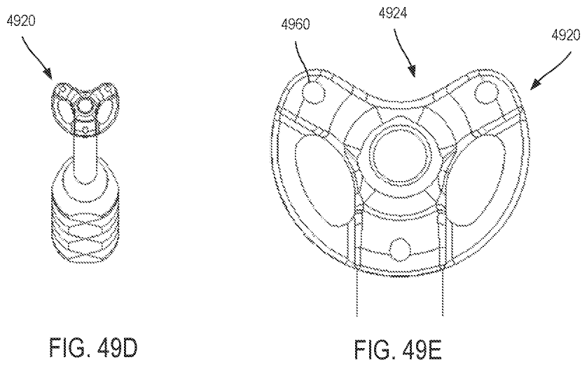

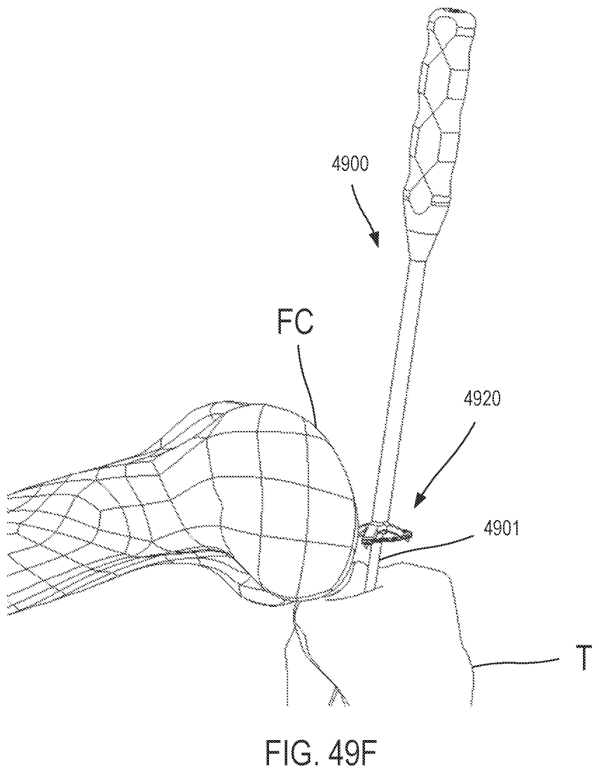

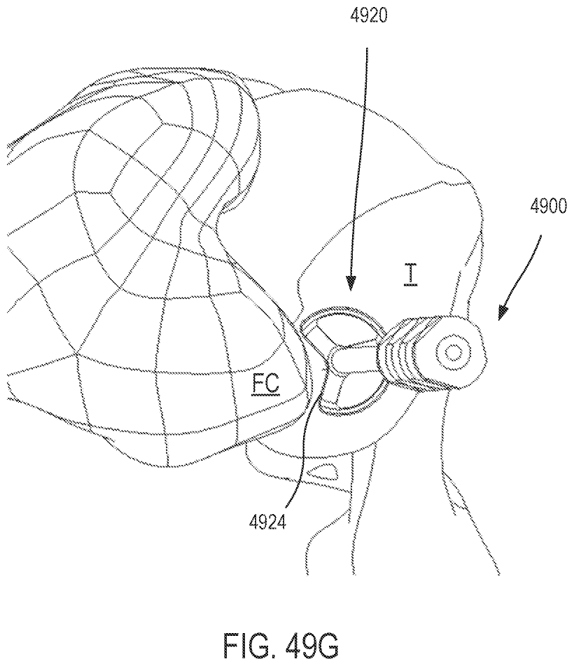

FIGS. 28A and 28B depict aspects of a tibial pin guide system 2800, according to embodiments of the present invention. Related aspects of tibial pin guide system embodiments are discussed elsewhere herein, for example in association with FIGS. 49A to 49G.

FIG. 29 depicts aspects of a tibial pin device 2900, according to embodiments of the present invention. The device can include a pin 2910. In some cases, tibial pin device 2900 can provide the same functionality and/or be implemented for the same intended use as the device depicted in one or more of FIGS. 49A to 49G.

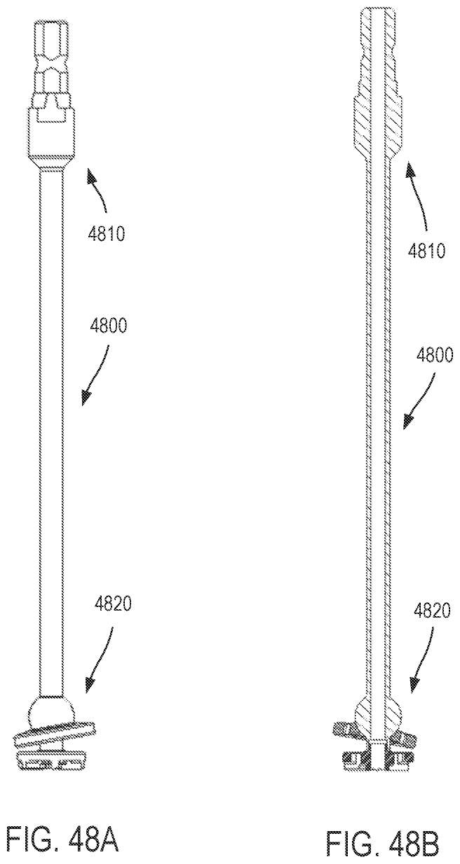

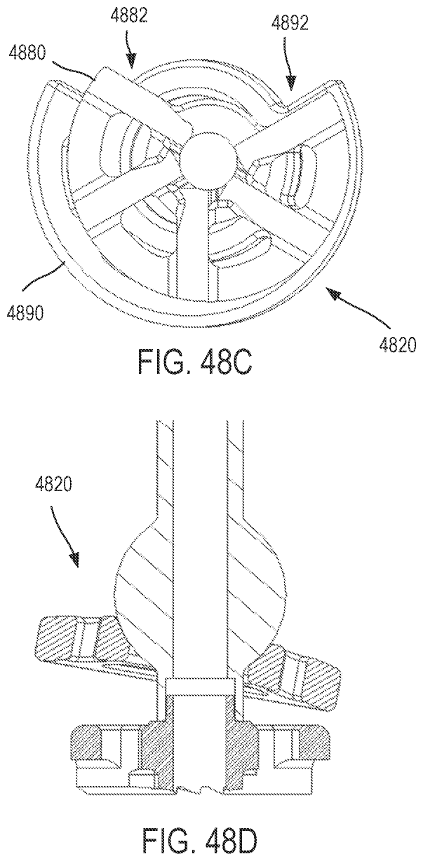

FIGS. 30A and 30B depict aspects of a tibial reamer (primary) system 3000, according to embodiments of the present invention. Aspects of related primary tibial reamer systems are discussed elsewhere herein, for example in association with FIGS. 48A to 48D.

FIGS. 31A and 31B depicts aspects of a tibial pin device 3100, according to embodiments of the present invention. The device can include a pin 3110. In some cases, tibial pin device 3100 can provide the same functionality and/or be implemented for the same intended use as the device depicted in one or more of FIGS. 48A to 48D.

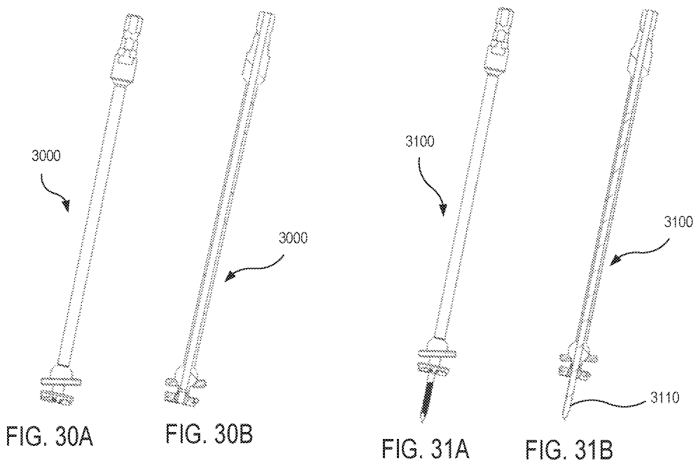

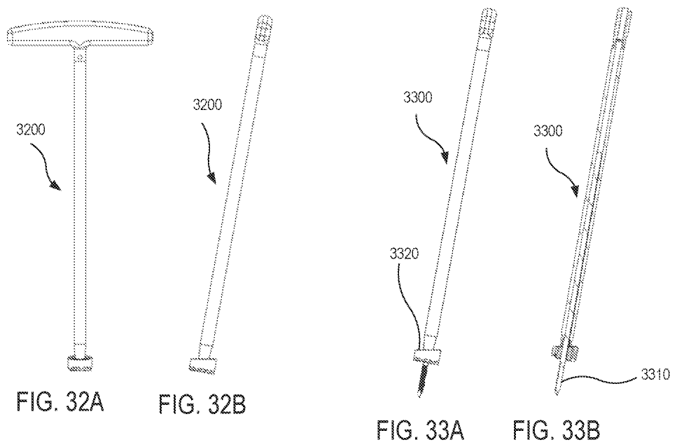

FIGS. 32A and 32B depict aspects of a tibial reamer (secondary) system 3200, according to embodiments of the present invention.

FIGS. 33A and 33B depict aspects of a tibial pin device 3300, according to embodiments of the present invention. The device can include a pin 3310. In some embodiments, a distal section 3320 of the device can be configured to mimic or match the geometry of the implant, while also functioning as a reaming/drilling/boring device. In some cases, the tibial pin device 3300 can be used after the primary reamer is used, to hand ream the bine for a precise implant fit.

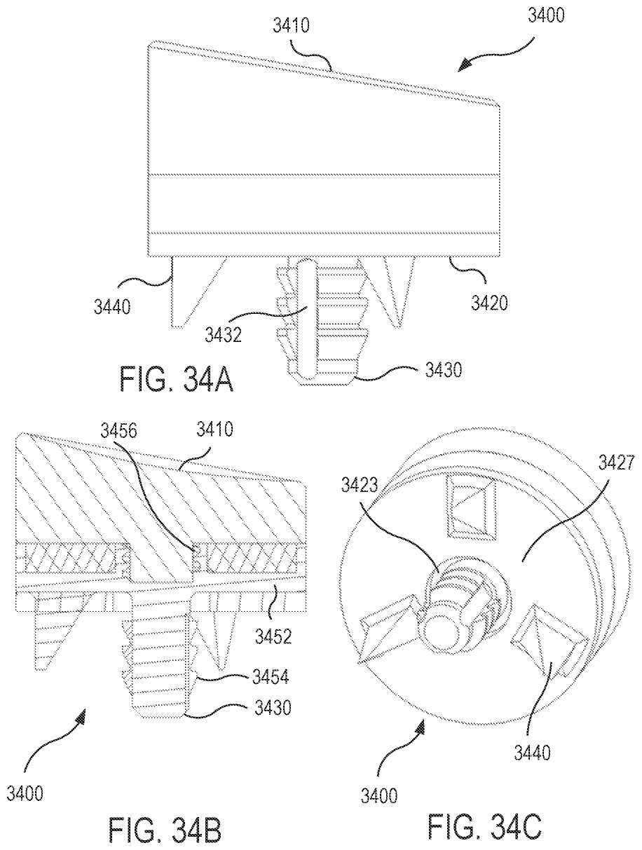

FIGS. 34A to 34C depict aspects of an exemplary tibial implant 3400 according to embodiments of the present invention. According to some embodiments, tibial implant 3400 includes a concave bearing surface portion 3410 and a subchondral surface portion 3420. Tibial implant 3400 can also include a peg 3430 and one or more anti-rotation spikes 3440. In this embodiment, a solid portion can surround a porous over-mold section. In some cases, the solid portion can include one or features such as a tray 3452, a spike 3440, and/or a post barb 3454. The implant can have a center thread 3456 to connect to a slap hammer removal tool. The peg 3430 can have one or more channels 3432, optionally which operate to allow for cement to flow. In some embodiments, cement is not used. The peg can be shorter and solid. The implant can include a pocket 3423 in a porous subchondral lattice 3427 at the base of the peg to contain overflowing cement.

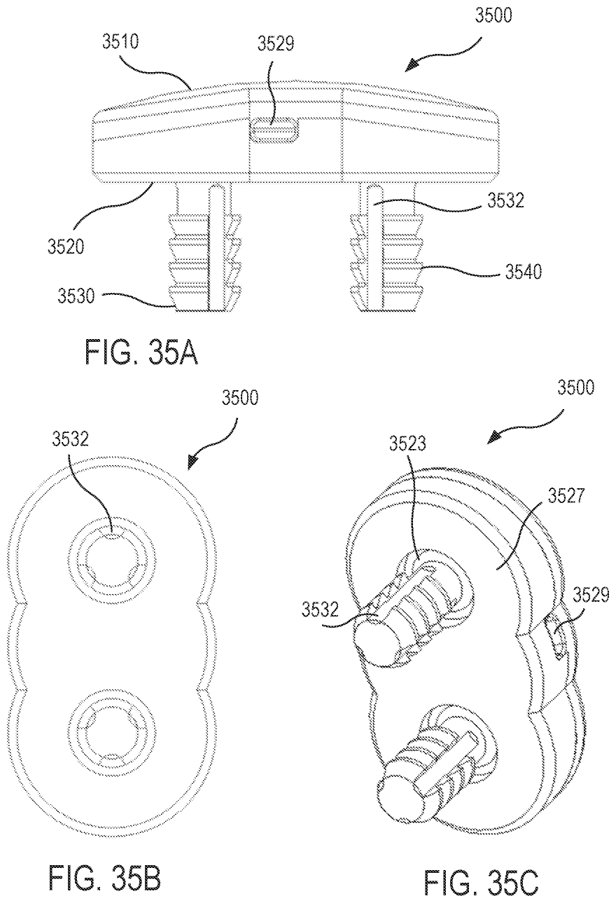

FIGS. 35A to 35C depict aspects of an exemplary femoral oblong implant 3500 according to embodiments of the present invention. Implant 3500 can include a convex bearing surface portion 3510 and a subchondral surface portion 3520. The femoral implant 3500 can also include one or more proximal pegs 3530. In some cases, the subchondral surface portion 3520 and/or one or more the proximal pegs 3530 can include a porous material, such as porous titanium. In the embodiment depicted here, proximal pegs 3530 include multiple barbs 3540. In some cases, the convex bearing surface portion 3510 of the femoral implant 3500 can include nonporous titanium. In some cases, the convex bearing surface portion 3510 can include nonporous stainless steel, nonporous cobalt chrome, nonporous ceramic, and the like. In some cases, the pegs 3530 and subchondral surface portion 3520 include a porous material, and the barbs 3540 and the convex bearing surface portion 3510 includes a solid or nonporous material. In some cases, the femoral implant can include no porous structure. That is, the femoral implant can be completely made of a solid or nonporous material. The convex bearing surface portion 3510 of the femoral implant 3500 has a three-circle or oblong profile. The three-circle profile provides a surface portion that has three sections, which allows for simple reaming with a circular reamer (e.g. in 3 adjacent reaming locations on the patient's bone, with overlapping circles). In some cases, the femoral implant 3500 is a monolithic unit. In this embodiment, the femoral implant 3500 includes no anti-rotation spikes, although such spikes may be present in other embodiments. In this embodiment, one or more pegs 3530 can have one or more channels 3532 that allow for cement to flow. In some cases, one or more pegs 3530 can be solid. In some cases, the implant 3500 can include one or more pocket 3523 in a porous subchondral lattice 3527 at the base of the peg to contain overflowing cement. In some cases, the implant 3500 can include a notch 3529 just below the solid section to allow for the connection of a removal tool.

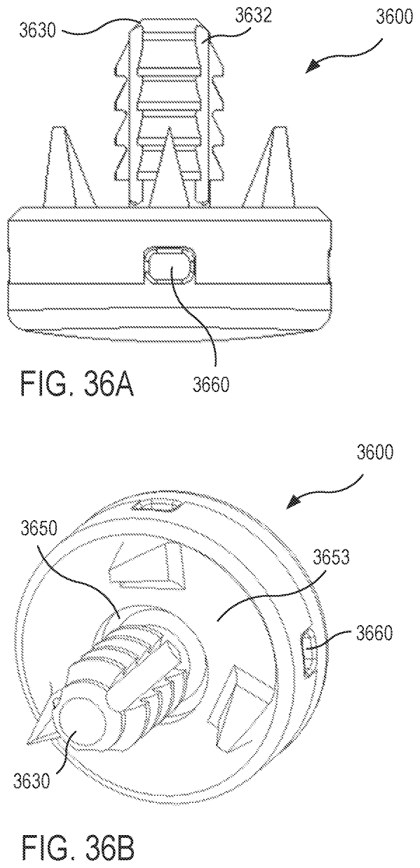

FIGS. 36A and 36B depict aspects of an exemplary femoral round implant 3600 according to embodiments of the present invention. In this embodiment, a peg 3630 can have one or more channels 3632 that allow for cement to flow. In some cases, the peg 3630 can be solid. In some cases, the implant 3600 can include a pocket 3650 in a porous subchondral lattice 3653 at the base of the peg 3630 to contain overflowing cement. In some cases, the implant 3600 can include a notch 3660 just below the solid section to allow for the connection of a removal tool.

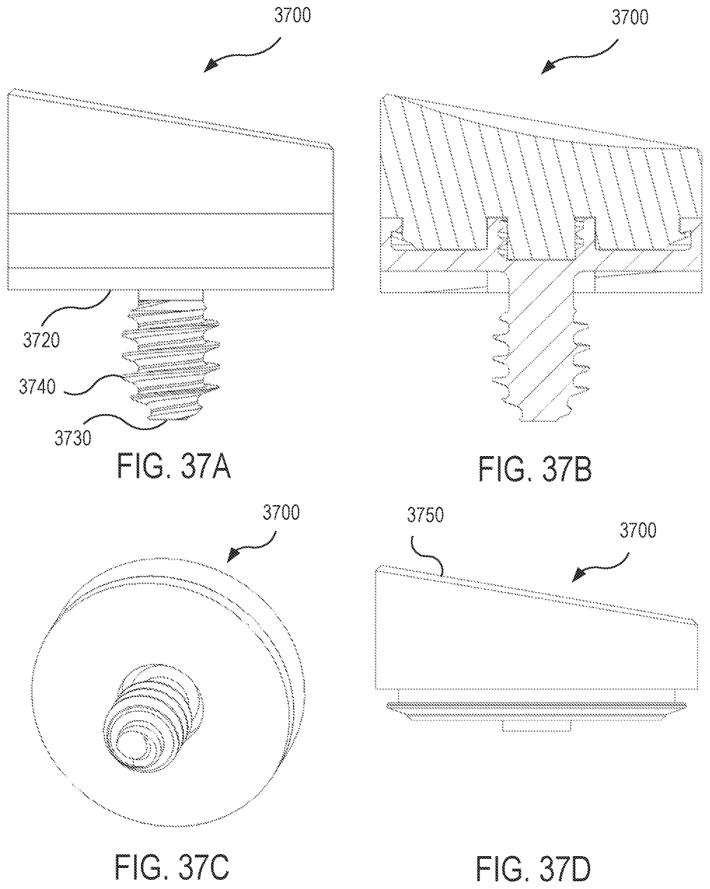

FIGS. 37A to 37D depict aspects of an exemplary tibial implant 3700 according to embodiments of the present invention. In this embodiment, the implant 3700 can have bone threads 3740 (e.g. on a peg 3730). A base 3720 of the implant 3700 can be driven in as one component by engaging with the threads 3740. For example, the threads 3740 can engage the bone of the patient. A tibial poly 3750 of the implant 3700 can snap into the base after being driven in.

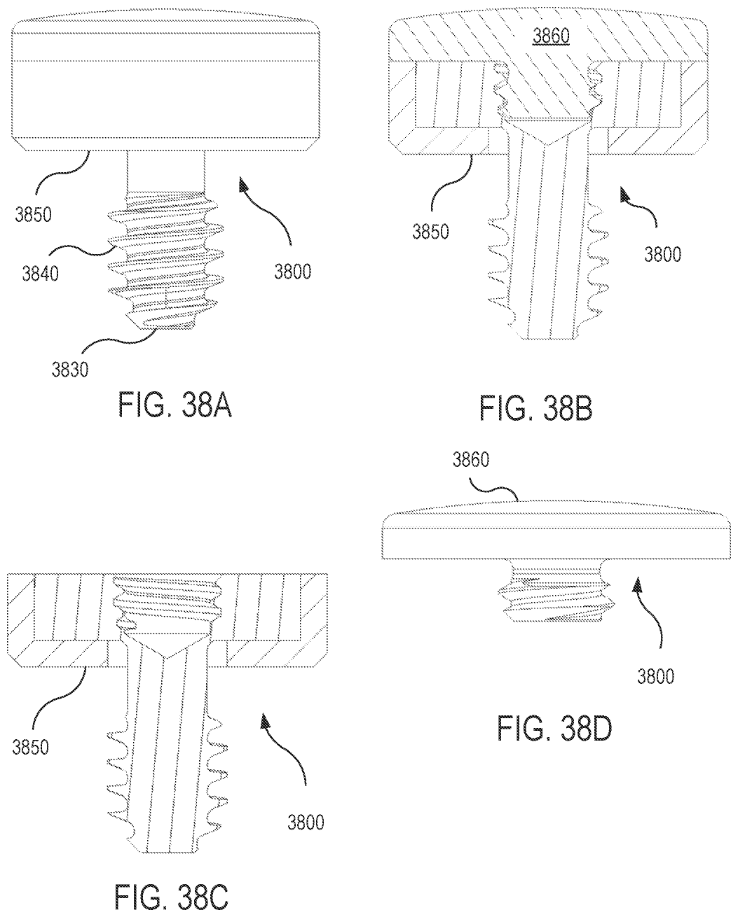

FIGS. 38A to 38D depict aspects of an exemplary femoral implant 3800 according to embodiments of the present invention. In this embodiment, the implant 3800 can have bone threads 3840 (e.g. on a peg 3830). A base 3850 of the implant 3800 can be driven into bone by engaging with the internal threads. For example, the threads 3840 can engage the bone of the patient. A bearing surface portion 3860 of the implant 3800 can thread into base 3850 using the same threads, or otherwise engage with the base 3850 via coupling.

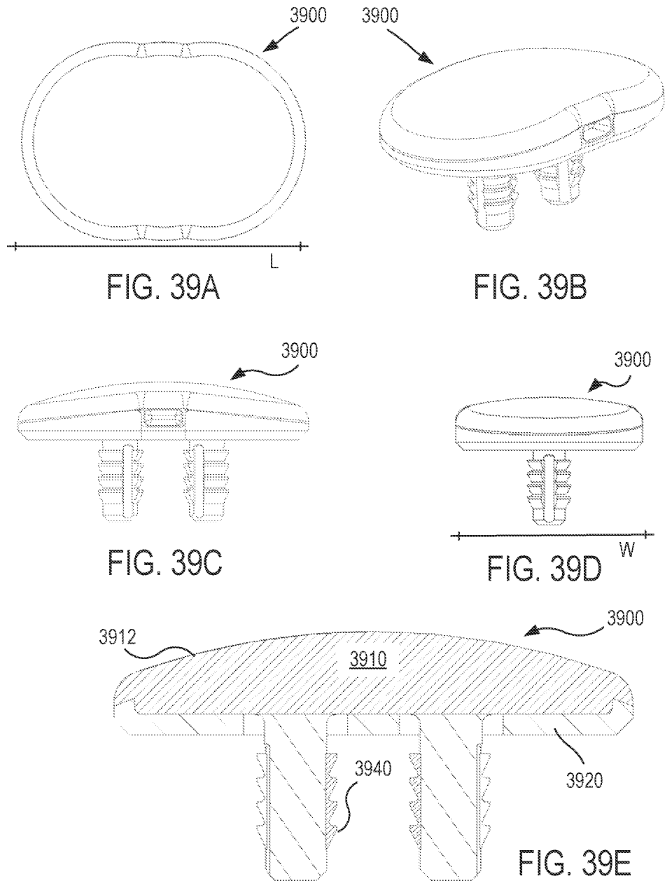

FIGS. 39A to 39E depict aspects of an exemplary femoral implant 3900 according to embodiments of the present invention. FIG. 39A provides a top plan view, FIG. 39B provides a perspective view, FIG. 39C provides a front view, FIG. 39D provides a right side view, and FIG. 39E provides a cross-section view. As shown here, femoral implant 3900 has an oblong shape. In some cases, implant 3900 can have a length L of about 25 mm and a width W of about 17.5 mm. As shown in FIG. 39E, the peg barbs 3940 and the convex surface portion 3910 of the implant 3900 can be made of a solid material, such as solid titanium. A subchondral surface portion 3920 of the implant 3900 can be made of a non-solid material, such as a random lattice, which may include a material such as titanium. In some cases, a bearing surface 3912 includes a titanium nitride (TiN) coating.

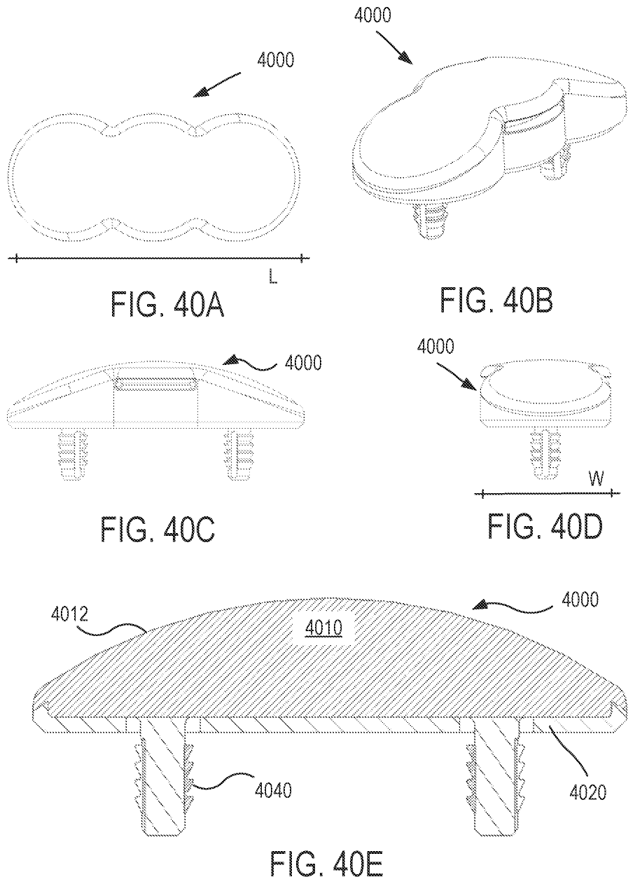

FIGS. 40A to 40E depict aspects of an exemplary femoral implant 4000 according to embodiments of the present invention. FIG. 40A provides a top plan view, FIG. 40B provides a perspective view, FIG. 40C provides a front view, FIG. 40D provides a right side view, and FIG. 40E provides a cross-section view. As shown here, femoral implant 4000 has an oblong shape. In some cases, implant 4000 can have a length L of about 40 mm and a width W of about 17.5 mm. As shown in FIG. 40E, the peg barbs 4040 and the convex surface portion 4010 of the implant 4000 can be made of a solid material, such as solid titanium. A subchondral surface portion 4020 of the implant 4000 can be made of a non-solid material, such as a random lattice, which may include a material such as titanium. In some cases, a bearing surface 4012 includes a titanium nitride (TiN) coating.

In some embodiments, a femoral oblong implant can have a width W having a value within a range from about 17.5 mm to about 27.5 mm. In some embodiments, a femoral oblong implant can have a length L having a value within a range from about 25 mm to about 40 mm. Exemplary width×length dimensions for a femoral oblong implant include 17.5 mm×25 mm, 17.5×30 mm, 17.5 mm×35 mm, 17.5 mm×40 mm, 20 mm×30 mm, 20 mm×35 mm, 20 mm×40 mm, 22.5 mm×30 mm, 22.5 mm×35 mm, 22.5 mm×40 mm, 25 mm×35 mm, 25 mm×40 mm, 27.5 mm×35 mm, 27.5 mm×40 mm, and the like.

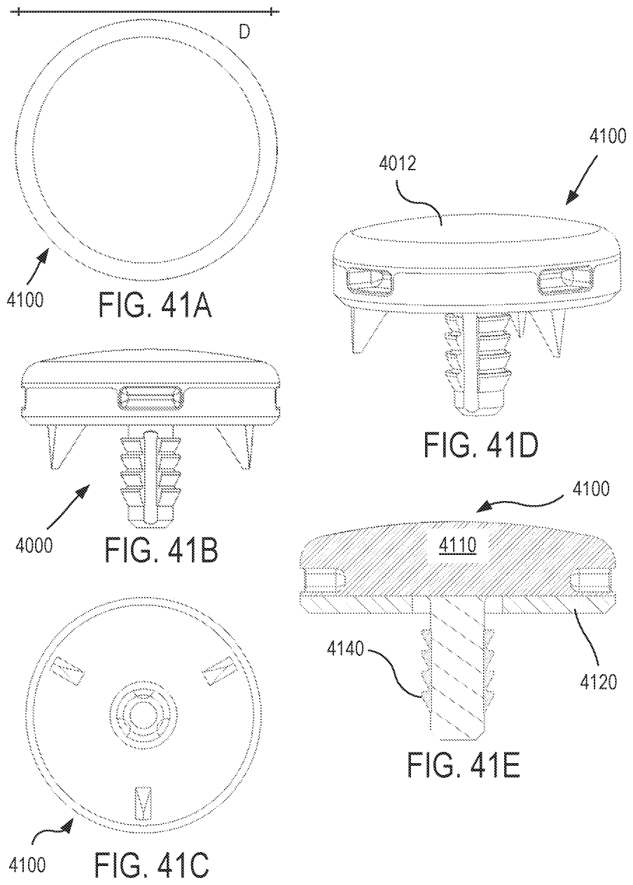

FIGS. 41A to 41E depict aspects of an exemplary femoral implant 4100 according to embodiments of the present invention. FIG. 41A provides a top plan view, FIG. 41B provides a side view, FIG. 41C provides a bottom plan view, FIG. 41D provides a perspective view, and FIG. 41E provides a cross-section view. As shown here, femoral implant 4100 has a round shape. In some cases, implant 4100 can have a diameter D of about 17.5 mm. As shown in FIG. 40E, the peg barbs 4140 and the convex surface portion 4110 of the implant 4100 can be made of a solid material, such as solid titanium. A subchondral surface portion 4120 of the implant 4100 can be made of a non-solid material, such as a random lattice, which may include a material such as titanium. In some cases, a bearing surface 4112 includes a titanium nitride (TiN) coating. In some embodiments, a femoral round implant can have a diameter D having a value within a range from about 17.5 mm to about 27.5 mm. Exemplary diameter dimensions for a femoral round implant include 17.5 mm, 20 mm, 22.5 mm, 25 mm, 27.5 mm, and the like.

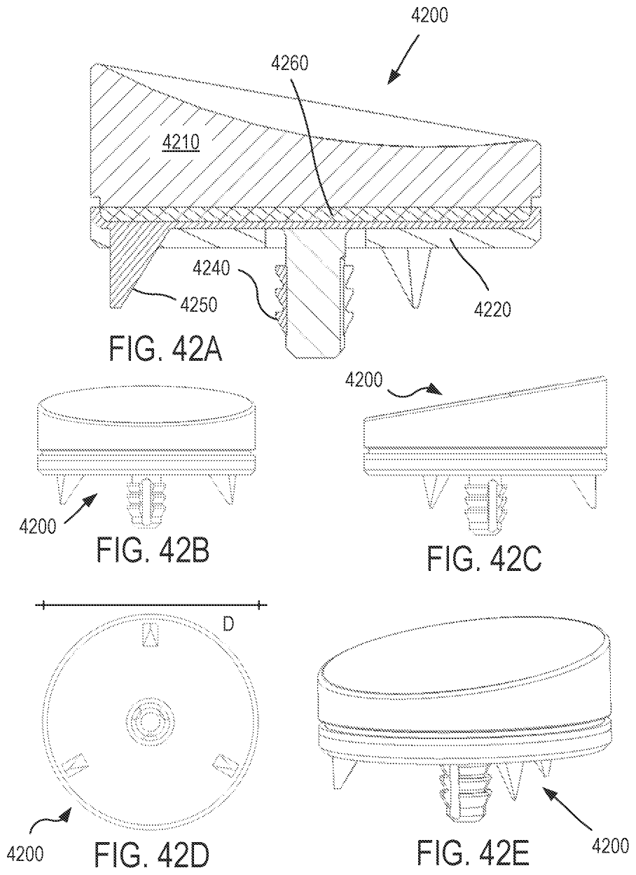

FIGS. 42A to 42E depict aspects of an exemplary tibial implant 4200 according to embodiments of the present invention. FIG. 42A provides a cross-section view, FIG. 42B provides a front side view, FIG. 42C provides a right side view, FIG. 42D provides a bottom plan view, and FIG. 42E provides a perspective. As shown here, tibial implant 4200 has a round shape (when viewed from the top or bottom). In some cases, implant 4200 can have a diameter D with a value within a range from about 15.0 mm to about 25.0 mm. As shown in FIG. 42A, the peg barbs 4240 and the anti-rotation spikes 4250 of the implant 4200 can be made of a solid material, such as solid titanium. A concave surface portion 4210 of the implant 4200 can be made of a plastic or polymer material, such as ultra-high-molecular-weight polyethylene (UHMWPE). A subchondral surface portion 4220 of the implant 4200 can be made of a non-solid material, such as a random lattice, which may include a material such as titanium. The implant 4200 can include an intermediate portion 4260 disposed between the concave surface portion 4210 and the subchondral surface portion 4220. In some cases, the intermediate portion 4260 can be made of a non-solid material, such as a diamond lattice, which may include a material such as titanium. In some embodiments, a tibial implant can have a diameter D having a value within a range from about 17.5 mm to about 25.0 mm. Exemplary diameter dimensions for a tibial implant include 17.5 mm, 20 mm, 25.0 mm, and the like. In some cases, the concave surface portion 4210 can be compression molded onto the subchondral surface portion 4220 or tibial tray.

FIGS. 43A to 43E depict aspects of a femoral primary reamer system 4300, according to embodiments of the present invention. FIG. 43A provides a side view, FIG. 43B provides a cross-section view, and FIG. 43C provides a perspective view. The primary femoral reamer system 4300 includes a proximal end 4310 and a distal end 4320, where the distal end 4320 is configured to ream bone of the patient, thereby producing a recess in the bone which is sized and/or configured to receive an implant. For example, the distal end 4320 can produce a recess or hole in the distal portion of the femur. FIG. 43D provides a bottom plan view, illustrating a distal end 4320 having a drill mechanism 4325, which may include cutting or boring elements. Drill mechanism 4325 has a diameter D, which can produce a bone recess or hole of similar diameter. FIG. 43E provides a cross-section view of the distal end 4320, illustrating a distal end 4320 having a washer mechanism 4370 that can operate to control the ream depth. In some cases, the washer mechanism 4370 can limit the ream depth by coming in contact with a condyle surface, for example located beyond a defect range. Related aspects of primary femoral reamer system embodiments are discussed elsewhere herein, for example in association with FIGS. 23A to 23E.

FIGS. 44A to 44E depict aspects of a secondary femoral reamer system 4400, according to embodiments of the present invention. FIG. 44A provides a side view, FIG. 44B provides a cross-section view, and FIG. 44C provides a perspective view. The secondary femoral reamer system 4400 includes a proximal end 4410 and a distal end 4420, where the distal end 4420 is configured to ream bone of the patient, thereby producing a recess in the bone which is sized and/or configured to receive an implant. For example, the distal end 4420 can produce a recess or hole in the distal portion of the femur. FIG. 44D provides a bottom plan view, illustrating a distal end 4420 having a drill mechanism 4425, which may include cutting or boring elements. Drill mechanism 4425 has a diameter D, which can produce a bone recess or hole of similar diameter. FIG. 44E provides a cross-section view of the distal end 4420. Related aspects of a secondary femoral reamer system embodiments are discussed elsewhere herein, for example in association with FIGS. 25A to 25E.

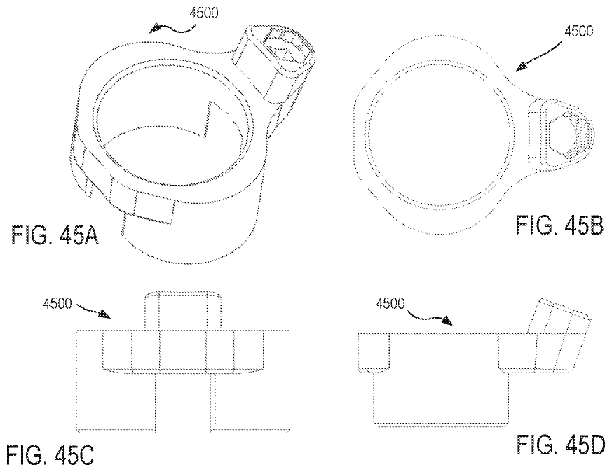

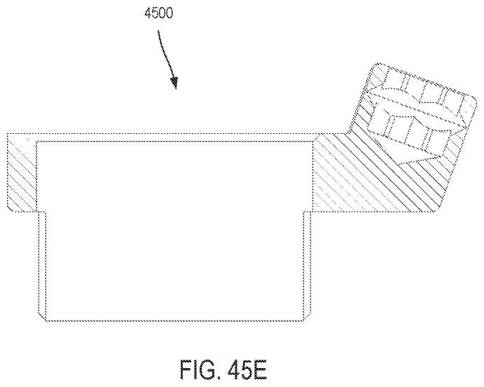

FIGS. 45A to 45E depict aspects of a reamer guide mechanism 4500, according to embodiments of the present invention. FIG. 45A provides a perspective view, FIG. 45B provides a top plan view, FIG. 45C provides a front side view, FIG. 45D provides a right side view, and FIG. 45E provides a cross-section view.

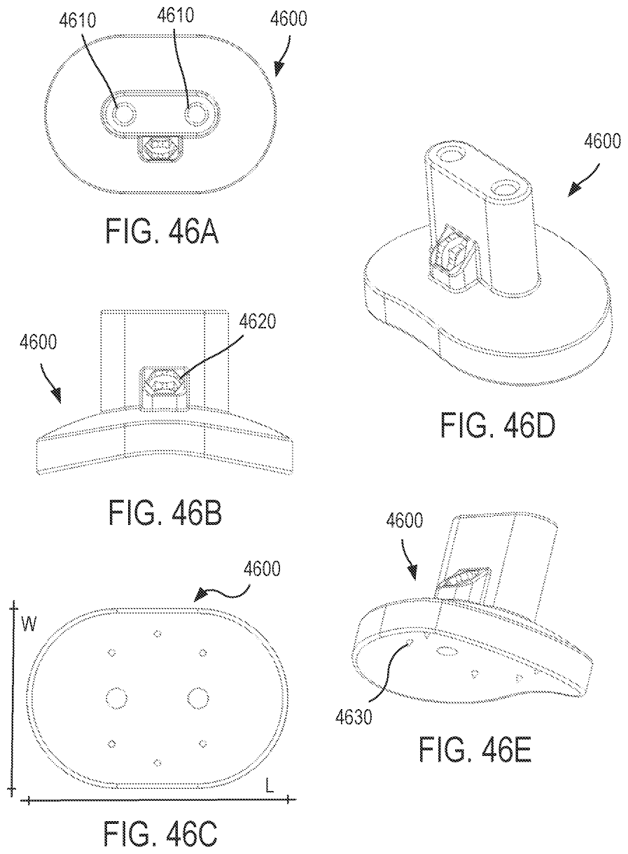

FIGS. 46A to 46E depict aspects of a pin guide sizer mechanism 4600, according to embodiments of the present invention. FIG. 46A provides a top plan view, FIG. 46B provides a front side view, FIG. 46C provides a bottom plan view, FIG. 46D provides an upper perspective view, and FIG. 46E provides a lower perspective view. As shown here, pin guide sizer mechanism 4600 includes pin guide holes 4610, a universal instrument handle socket 4620, and spikes 4630 which can operate to stabilize the guide mechanism 4600 while installing pins (e.g. by engaging patient bone). In some cases, the pin guide mechanism 4600 can have a width W and a length L that can be used to determine the size of the implant. In some cases, the length and width offerings are 1 to 1 with implant sizing, or otherwise mimic or match the implant sizing.

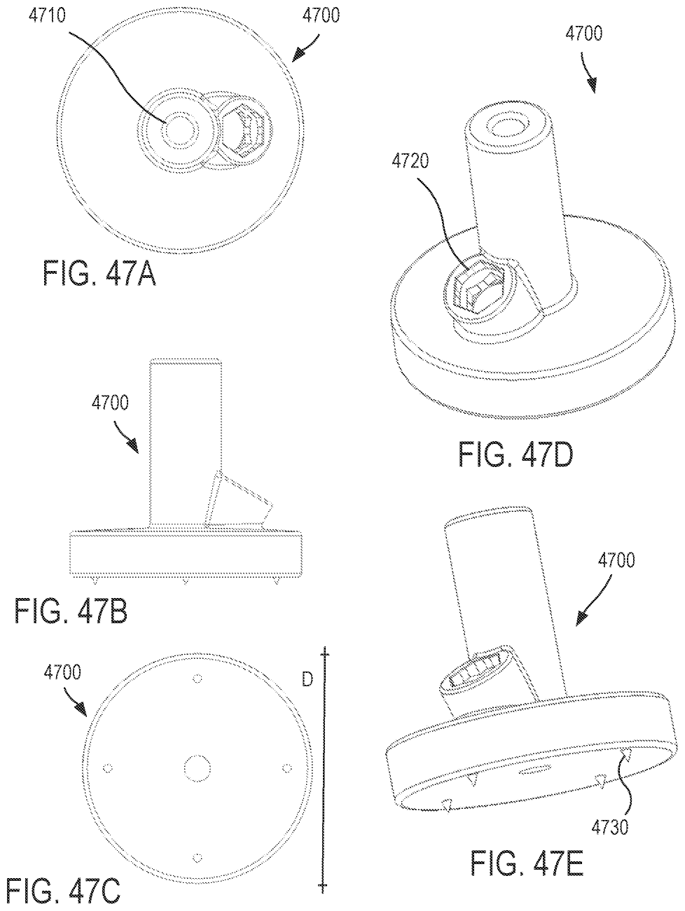

FIGS. 47A to 47E depict aspects of a pin guide sizer mechanism 4700, according to embodiments of the present invention. FIG. 47A provides a top plan view, FIG. 47B provides a right side view, FIG. 47C provides a bottom plan view, FIG. 47D provides an upper perspective view, and FIG. 47E provides a lower perspective view. As shown here, pin guide sizer mechanism 4700 includes a pin guide hole 4710, a universal instrument handle socket 4720, and spikes 4730 which can operate to stabilize the guide mechanism 4700 while installing pins (e.g. by engaging patient bone). In some cases, the pin guide mechanism 4700 can have a diameter D that can be used to determine the size of the implant.

FIGS. 48A to 48D depict aspects of a primary tibial reamer system 4800, according to embodiments of the present invention. FIG. 48A provides a side view and FIG. 48B provides a cross-section view. The primary tibial reamer system 4800 includes a proximal end 4810 and a distal end 4820, where the distal end 4820 is configured to ream bone of the patient, thereby producing a recess in the bone which is sized and/or configured to receive an implant. For example, the distal end 4820 can produce a recess or hole in the distal portion of the femur. FIG. 48C provides a bottom plan view and FIG. 48D provides a cross-section view. As depicted in FIG. 48C, the distal end 4820 of the primary tibial reamer system can include a reamer head 4880 having a cutout 4882 and a depth stop washer 4890 having a cutout 4892. The cutouts 4882, 4892 can enable the distal end 4820 to clear a femoral condyle of the patient. Aspects of such femoral clearance techniques are discussed elsewhere herein, for example in association with FIGS. 49F and 49G.

FIGS. 49A to 49G depict aspects of a tibial pin guide system 4900 and related methods of use, according to embodiments of the present invention. FIGS. 49A and 49B provide side views, and FIG. 49C provides a cross-section view. A tibial pin guide system 4900 can include a proximal end 4910 and a distal end 4920. As shown in FIG. 49C, a distal end of the tibial pin guide system 4900 can define a distal engagement plane 4928 and an interior lumen 4902 of the tibial guide system 4900 can define a central longitudinal axis 4904, such that a pin delivered through the lumen 4902 to the patient can enter the bone at a pin angle A, where angle A is the angle between a vector V normal to the plane 4928 and the central longitudinal axis 4904. As shown in FIG. 49E, the distal end 4920 of the tibial guide system 4900 includes a cutout 4924, which can enable the system 4900 to clear a femoral condyle. The distal end 4920 also includes spikes 4960 which can operate to stabilize the system 4900 during use while installing pins. For example, the spikes 4960 can engage patient bone. FIGS. 49F and 49G illustrate aspects of a method during which system 4900 is used to deliver a pin 4901 to a patient bone (e.g. tibia T). As shown here, a cutout 4924 of the distal end 4920 of the system can enable the distal end to clear a femoral condyle FC. FIG. 49F shows a side view of a patient knee, and FIG. 49G shows a superior view of a patient knee.

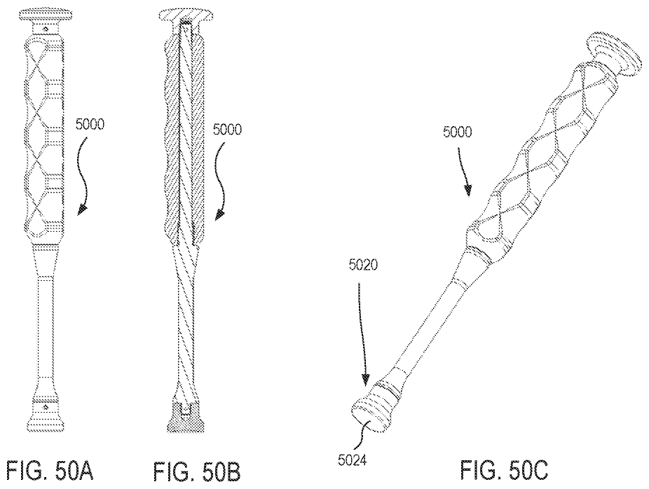

FIGS. 50A to 50C depict aspects of an impactor system 5000, according to embodiments of the present invention. FIG. 50A provides a side view, FIG. 50B provides a cross-section view, and FIG. 50C provides a perspective view. As shown here, an impactor system 5000 can include a distal end 5020 having a concave surface 5024, which can be shaped or configured to match or complement a convex surface of an implant (e.g. a femoral implant).

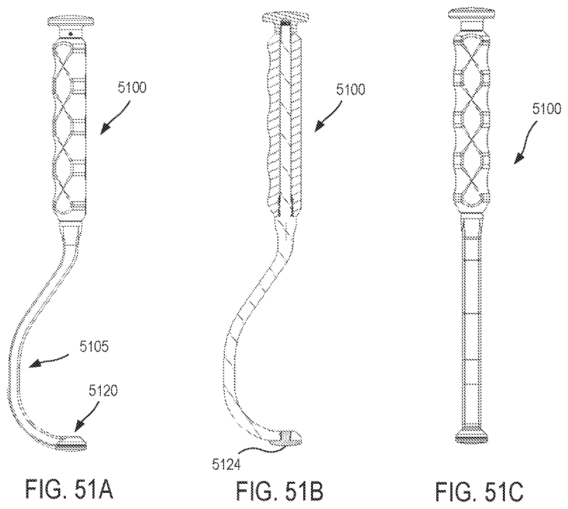

FIGS. 51A to 51C depict aspects of an impactor system 5100, according to embodiments of the present invention. FIG. 51A provides a side view, FIG. 51B provides a cross-section view, and FIG. 51C provides a front view. As shown here, an impactor system 5100 can include a distal end 5120 having a convex surface 5124, which can be shaped or configured to match or complement a concave surface of an implant (e.g. a tibial implant). Impactor system 5100 can also include a distal curve 5105, which can enable the impactor system 5100 to clear a femoral condyle of the patient. Aspects of such femoral clearance techniques are discussed elsewhere herein, for example in association with FIGS. 49F and 49G.

Although the preceding description contains significant detail in relation to certain preferred embodiments, it should not be construed as limiting the scope of the invention but rather as providing illustrations of the preferred embodiments.

Embodiments of the present invention encompass kits having arthroplasty resurfacing system as disclosed herein. In some embodiments, the kit includes one or more arthroplasty resurfacing system implants and/or insertion devices, along with instructions for using the device(s) for example according to any of the methods disclosed herein.

All features of the described systems and devices are applicable to the described methods mutatis mutandis, and vice versa.

Although the foregoing invention has been described in some detail by way of illustration and example for purposes of clarity of understanding, one of skill in the art will appreciate that certain changes, modifications, alternate constructions, and/or equivalents may be practiced or employed as desired, and within the scope of the appended claims. In addition, each reference provided herein in incorporated by reference in its entirety to the same extent as if each reference were individually incorporated by reference. Relatedly, all publications, patents, patent applications, journal articles, books, technical references, and the like mentioned in this specification are herein incorporated by reference to the same extent as if each individual publication, patent, patent application, journal article, book, technical reference, or the like was specifically and individually indicated to be incorporated by reference.

1. A unicompartmental knee arthroplasty resurfacing system, comprising:
a femoral implant comprising a convex bearing surface portion and a subchondral surface portion; and
a tibial implant comprising a concave bearing surface portion and a subchondral surface portion.

2. The unicompartmental knee arthroplasty resurfacing system according to claim 1,
wherein the concave bearing surface portion of the tibial implant has a rim that defines a proximal plane,
wherein the subchondral surface portion of the tibial implant defines a distal plane,
and wherein the proximal plane defined by the rim of the concave bearing surface portion of the tibial implant is non-parallel to the distal plane defined by the subchondral surface portion of the tibial implant.

3. The unicompartmental knee arthroplasty resurfacing system according to claim 1,
wherein the concave bearing surface portion of the tibial implant has a rim that defines a proximal plane,
wherein the subchondral surface portion of the tibial implant defines a distal plane,
and wherein the proximal plane defined by the rim of the concave bearing surface portion of the tibial implant is parallel to the distal plane defined by the subchondral surface portion of the tibial implant.

4. The unicompartmental knee arthroplasty resurfacing system according to claim 1, wherein the femoral implant further comprises at least one proximal peg.

5. (canceled)

6. The unicompartmental knee arthroplasty resurfacing system according to claim 1, wherein the femoral implant further comprises at least one anti-rotation spike.

7. (canceled)

8. (canceled)

9. The unicompartmental knee arthroplasty resurfacing system according to claim 1,
wherein the concave bearing surface portion of the tibial implant has a rim that defines a proximal plane,
wherein the subchondral surface portion of the tibial implant defines a distal plane,
wherein the tibial implant further comprises at least one distal peg, and
wherein the at least one distal peg of the tibial implant defines an axis that is perpendicular to the distal plane defined by the subchondral surface portion of the tibial implant and that is non perpendicular to the proximal plane defined by the rim of the concave bearing surface portion of the tibial implant.

10.-24. (canceled)

10. The unicompartmental knee arthroplasty resurfacing system according to claim 1, wherein the convex bearing surface portion of the femoral implant comprises a three-circle profile.

26.-39. (canceled)

11. An arthroplasty resurfacing system, comprising:
a first implant comprising a convex bearing surface portion and a subchondral surface portion; and
a second implant comprising a concave bearing surface portion and a subchondral surface portion,
wherein the system is configured for implantation into a joint of a patient.

41. (canceled)

12. The arthroplasty resurfacing system according to claim 40,
wherein the first implant comprises a femoral implant, the second implant comprises a tibial implant, and the joint comprises a knee joint.

13. The arthroplasty resurfacing system according to claim 40,
wherein the concave bearing surface portion of the second implant has a rim that defines a proximal plane,
wherein the subchondral surface portion of the second implant defines a distal plane,
and wherein the proximal plane defined by the rim of the concave bearing surface portion of the second implant is non-parallel to the distal plane defined by the subchondral surface portion of the second implant.

14. The arthroplasty resurfacing system according to claim 40,
wherein the concave bearing surface portion of the second implant has a rim that defines a proximal plane,
wherein the subchondral surface portion of the second implant defines a distal plane,
and wherein the proximal plane defined by the rim of the concave bearing surface portion of the second implant is parallel to the distal plane defined by the subchondral surface portion of the second implant.

15. The arthroplasty resurfacing system according to claim 40, wherein the first implant further comprises at least one proximal peg.

46. (canceled)

16. The arthroplasty resurfacing system according to claim 40, wherein the first implant further comprises at least one anti-rotation spike.

48. (canceled)

49. (canceled)

17. The arthroplasty resurfacing system according to claim 40,
wherein the concave bearing surface portion of the second implant has a rim that defines a proximal plane,
wherein the subchondral surface portion of the second implant defines a distal plane,
wherein the second implant further comprises at least one distal peg, and
wherein the at least one distal peg of the second implant defines an axis that is perpendicular to the distal plane defined by the subchondral surface portion of the second implant and that is non perpendicular to the proximal plane defined by the rim of the concave bearing surface portion of the second implant.

51.-80. (canceled)

18. A method of implanting an arthroplasty resurfacing system into a joint of a patient, comprising:
engaging a first implant of the resurfacing system with a distal portion of a first bone of the joint of the patient, the first implant comprising a convex bearing surface portion and a subchondral surface portion; and
engaging a second implant of the resurfacing system with a proximal portion of a second bone of the joint of the patient, the second implant comprising a concave bearing surface portion and a subchondral surface portion.

82. (canceled)

19. The method according to claim 81, wherein the joint comprises a knee joint, the first implant comprises a femoral implant, and the second implant comprises a tibial implant.

20. The method according to claim 81, wherein the concave bearing surface portion of the second implant has a rim that defines a proximal plane,
wherein the subchondral surface portion of the second implant defines a distal plane,
and wherein the proximal plane defined by the rim of the concave bearing surface portion of the second implant is non-parallel to the distal plane defined by the subchondral surface portion of the second implant.

21. The method according to claim 81,
wherein the concave bearing surface portion of the second implant has a rim that defines a proximal plane,
wherein the subchondral surface portion of the second implant defines a distal plane,
and wherein the proximal plane defined by the rim of the concave bearing surface portion of the second implant is parallel to the distal plane defined by the subchondral surface portion of the second implant.

22. The arthroplasty resurfacing system according to claim 81, wherein the first implant further comprises at least one proximal peg.

87. (canceled)

88. The arthroplasty resurfacing system according to claim 81, wherein the first implant further comprises at least one anti-rotation spike.

89.-121. (canceled)

122. The unicompartmental knee arthroplasty resurfacing system according to claim 1, wherein the tibial implant further comprises at least one anti-rotation spike.

123. The unicompartmental knee arthroplasty resurfacing system according to claim 1,
wherein the subchondral surface portion of the tibial implant has a proximal side and a distal side, and
wherein the concave bearing surface portion of the tibial implant is compression molded with the proximal side of the subchondral surface portion of the tibial implant.

124. The unicompartmental knee arthroplasty resurfacing system according to claim 1, wherein the convex bearing surface portion of the femoral implant comprises a round profile.

125. The unicompartmental knee arthroplasty resurfacing system according to claim 2,
wherein the tibial implant has an anterior portion and a posterior portion, and
wherein a distance between the proximal plane and the distal plane at the anterior portion is greater than a distance between the proximal plane and the distal plane at the posterior portion.

126. The method according to claim 81,
wherein the joint is a knee joint, the first bone is a femur, the second bone is a tibia, the first implant is a femoral implant, and the second implant is a tibial implant,
wherein the concave bearing surface portion of the tibial implant has a rim that defines a proximal plane, the subchondral surface portion of the tibial implant defines a distal plane, and the proximal plane defined by the rim of the concave bearing surface portion of the tibial implant is non-parallel to the distal plane defined by the subchondral surface portion of the tibial implant,
wherein the tibial implant has an anterior portion and a posterior portion, and wherein a distance between the proximal plane and the distal plane at the anterior portion is greater than a distance between the proximal plane and the distal plane at the posterior portion, and
wherein the engaging step for the tibial implant comprises advancing the tibial implant from location proximal to the proximal portion of the tibia toward the proximal portion of the tibia.

127. The unicompartmental knee arthroplasty resurfacing system according to claim 123,
wherein the proximal side of the subchondral surface portion comprises a porous material and the distal side of the subchondral surface portion comprises an irregular lattice, and
wherein the concave bearing surface portion of the tibial implant is compression molded with the porous material of the proximal side of the subchondral surface portion.
